(19) United States
(12) Patent Application Publication
(10) Pub. No.: US 2018/0273525 A1
(43) Pub. Date: Sep. 27, 2018

(54) Fused Pyridine Derivatives As Kinase Inhibitors

(71) Applicants: UCB Biopharma SPRL, Brussels (BE); Katholieke Universiteit Leuven, K.U.Leuven R&D, Leuven (BE)

(72) Inventors: Helen Tracey Horsley, Slough, Berkshire (GB); James Madden, Abingdon, Oxfordshire (GB); James Thomas Reuberson, Slough, Berkshire (GB); Julian Hugh Rowley, Abingdon, Oxfordshire (GB)

(21) Appl. No.: 15/762,454

(22) PCT Filed: Sep. 28, 2016
(86) PCT No.: PCT/EP2016/073029

§ 371 Date: Mar. 22, 2018

(30) Foreign Application Priority Data
Sep. 30, 2015 (GB) 1517264.6

(51) Int. Cl. C07D 471/04; A61P 31/12; A61P 33/06; A61P 37/06; A61P 29/00; A61P 35/00

(52) U.S. Cl. CPC C07D 471/04; A61P 31/12; A61P 33/06; A61K 2121/00; A61P 29/00; A61P 35/00; A61P 37/06

(57) ABSTRACT

A series of substituted pyrido[3,2-d]pyrimidine and 1,5-naphthyridine derivatives of formula (I), as defined herein, being selective inhibitors of phosphatidylinositol-4-kinase IIIβ (PI4KIIIβ) activity, are beneficial in the treatment and/or prevention of various human ailments, including inflammatory, autoimmune and oncological disorders; viral diseases and malaria; and organ and cell transplant rejection.

The present invention relates to a class of fused pyridine derivatives, and to their use in therapy. More particularly, the present invention provides substituted pyrido[3,2-d]pyrimidine and 1,5-naphthyridine derivatives. These compounds are selective inhibitors of phosphatidylinositol-4-kinase IIIβ (PI4KIIIβ) activity, and are accordingly of benefit as pharmaceutical agents, especially in the treatment of adverse inflammatory, autoimmune and oncological disorders, in the treatment of viral diseases and malaria, and in the management of organ and cell transplant rejection.

In addition, the compounds in accordance with the present invention may be beneficial as pharmacological standards for use in the development of new biological tests and in the search for new pharmacological agents. Thus, the compounds of this invention may be useful as radioligands in assays for detecting pharmacologically active compounds.

WO 2013/034738 discloses that inhibitors of PI4KIIIβ activity are useful as medicaments for the treatment of autoimmune and inflammatory disorders, and organ and cell transplant rejection.

Inhibitors of PI4KIIIβ have been identified as molecules with an ideal activity profile for the prevention, treatment and elimination of malaria (cf. C. W. McNamara et al., Nature, 2013, 504, 248-253).

WO 2010/103130 describes a family of oxazolo[5,4-d]pyrimidine, thiazolo[5,4-d]-pyrimidine, thieno[2,3-d]pyrimidine and purine derivatives that are active in a range of assays, including the Mixed Lymphocyte Reaction (MLR) test, and are stated to be effective for the treatment of immune and autoimmune disorders, and organ and cell transplant rejection. WO 2011/147753 discloses the same family of compounds as having significant antiviral activity. Furthermore, WO 2012/035423 discloses the same family of compounds as having significant anticancer activity.

WO 2013/024291, WO 2013/068458, WO 2014/053581 and WO 2014/096423 describe various series of fused pyrimidine derivatives that are stated to be of benefit as pharmaceutical agents, especially in the treatment of adverse inflammatory, autoimmune and oncological disorders, in the treatment of viral diseases, and in the management of organ and cell transplant rejection.

Copending international patent applications PCT/EP2015/063048, PCT/EP2015/063051 and PCT/EP2015/063052 (published on 23 Dec. 2015 as WO 2015/193167, WO 2015/193168 and WO 2015/193169 respectively) describe various series of fused bicyclic heteroaromatic derivatives that are stated to be selective inhibitors of PI4KIIIβ activity, and accordingly of benefit as pharmaceutical agents, especially in the treatment of adverse inflammatory, autoimmune and oncological disorders, in the treatment of viral diseases, and in the management of organ and cell transplant rejection.

Various classes of substituted fused bicyclic heteroaromatic compounds that are stated to be selective PI4KIIIβ inhibitors, and to exhibit antiviral activity, are described in the scientific literature (cf. I. Mejdrová et al., J. Med. Chem., 2015, 58, 3767-3793; A. M. MacLeod et al., ACS Med. Chem. Lett., 2013, 4, 585-589; and M. Arita et al., J. Virol., 2011, 85, 2364-2372).

None of the prior art available to date, however, discloses or suggests the precise structural class of fused pyridine derivatives as provided by the present invention as having activity as PI4KIIIβ inhibitors.

The compounds of the present invention are potent and selective inhibitors of PI4KIIIβ activity, inhibiting the kinase affinity of human PI4KIIIβ (IC₅₀) at concentrations of 50 μM or less, generally of 20 μM or less, usually of 5 μM or less, typically of 1 μM or less, suitably of 500 nM or less, ideally of 100 nM or less, and preferably of 20 nM or less (the skilled person will appreciate that a lower IC₅₀ figure denotes a more active compound). The compounds of the invention may possess at least a 10-fold selective affinity, typically at least a 20-fold selective affinity, suitably at least a 50-fold selective affinity, and ideally at least a 100-fold selective affinity, for human PI4KIIIβ relative to other human kinases.

Certain compounds in accordance with the present invention are active as inhibitors when subjected to the Mixed Lymphocyte Reaction (MLR) test. The MLR test is predictive of immunosuppression or immunomodulation. Thus, when subjected to the MLR test, certain compounds of the present invention display an IC₅₀ value of 10 μM or less, generally of 5 μM or less, usually of 2 μM or less, typically of 1 μM or less, suitably of 500 nM or less, ideally of 100 nM or less, and preferably of 20 nM or less (again, the skilled person will appreciate that a lower IC₅₀ figure denotes a more active compound).

The present invention provides a compound of formula (I) or an N-oxide thereof, or a pharmaceutically acceptable salt or solvate thereof:

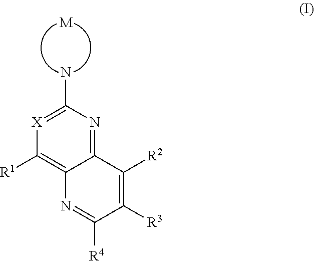

wherein

X represents N or CH;

M represents the residue of an optionally substituted saturated four-, five-, six- or seven-membered monocyclic ring containing one nitrogen atom and 0, 1, 2 or 3 additional heteroatoms independently selected from N, 0 and S, but containing no more than one O or S atom; or

M represents the residue of an optionally substituted saturated or unsaturated 5- to 10-membered fused bicyclic ring system containing one nitrogen atom and 0, 1, 2 or 3 additional heteroatoms independently selected from N, O and S, but containing no more than one O or S atom; or

M represents the residue of an optionally substituted saturated 5- to 9-membered bridged bicyclic ring system containing one nitrogen atom and 0, 1, 2 or 3 additional heteroatoms independently selected from N, O and S, but containing no more than one O or S atom; or

M represents the residue of an optionally substituted saturated 5- to 9-membered spirocyclic ring system containing one nitrogen atom and 0, 1, 2 or 3 additional heteroatoms independently selected from N, O and S, but containing no more than one O or S atom;

R¹, R² and R³ independently represent hydrogen, halogen, cyano, nitro, hydroxy, trifluoromethyl, trifluoromethoxy, —OR^{a}, —SR^{a}, —SOR^{a}, —SO₂R^{a}, —NR^{b}R^{c}, —CH₂NR^{b}R^{c}, —NR^{c}COR^{d}, —CH₂NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b} or —SO₂NR^{b}R^{c}; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, heteroaryl or heteroaryl(C₁₋₆)alkyl, any of which groups may be optionally substituted by one or more substituents;

R⁴ represents hydrogen, halogen, cyano, trifluoromethyl or C₁₋₆ alkyl;

R^{a} represents hydrogen; or R^{a} represents C₁₋₆ alkyl, aryl, aryl(C₁₋₆)alkyl, heteroaryl or heteroaryl(C₁₋₆)alkyl, any of which groups may be optionally substituted by one or more substituents;

R^{b} and R^{c} independently represent hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or

R^{b} and R^{c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;

R^{d} represents hydrogen; or C₁₋₆ alkyl, C₃₋₇ cycloalkyl, aryl, C₃₋₇ heterocycloalkyl or heteroaryl, any of which groups may be optionally substituted by one or more substituents; and

R^{e} represents C₁₋₆ alkyl, aryl or heteroaryl, any of which groups may be optionally substituted by one or more substituents.

Where any of the groups in the compounds of formula (I) above is stated to be optionally substituted, this group may be unsubstituted, or substituted by one or more substituents. Typically, such groups will be unsubstituted, or substituted by one or two substituents.

For use in medicine, the salts of the compounds of formula (I) will be pharmaceutically acceptable salts. Other salts may, however, be useful in the preparation of the compounds of the invention or of their pharmaceutically acceptable salts. Suitable pharmaceutically acceptable salts of the compounds of this invention include acid addition salts which may, for example, be formed by mixing a solution of the compound of the invention with a solution of a pharmaceutically acceptable acid such as hydrochloric acid, sulfuric acid, methanesulfonic acid, fumaric acid, maleic acid, succinic acid, acetic acid, benzoic acid, citric acid, tartaric acid or phosphoric acid. Furthermore, where the compounds of the invention carry an acidic moiety, e.g. carboxy, suitable pharmaceutically acceptable salts thereof may include alkali metal salts, e.g. sodium or potassium salts; alkaline earth metal salts, e.g. calcium or magnesium salts; and salts formed with suitable organic ligands, e.g. quaternary ammonium salts.

The present invention includes within its scope solvates of the compounds of formula (I) above. Such solvates may be formed with common organic solvents, e.g. hydrocarbon solvents such as benzene or toluene; chlorinated solvents such as chloroform or dichloromethane; alcoholic solvents such as methanol, ethanol or isopropanol; ethereal solvents such as diethyl ether or tetrahydrofuran; or ester solvents such as ethyl acetate. Alternatively, the solvates of the compounds of formula (I) may be formed with water, in which case they will be hydrates.

Suitable alkyl groups which may be present on the compounds of the invention include straight-chained and branched C₁₋₆ alkyl groups, for example C₁₋₄ alkyl groups. Typical examples include methyl and ethyl groups, and straight-chained or branched propyl, butyl, pentyl and hexyl groups. Particular alkyl groups include methyl, ethyl, n-propyl, isopropyl, n-butyl, sec-butyl, isobutyl, tert-butyl, 2,2-dimethylpropyl and 3-methylbutyl. Derived expressions such as “C₁₋₆ alkoxy”, “C₁₋₆ alkylthio”, “C₁₋₆ alkylsulfonyl” and “C₁₋₆ alkylamino” are to be construed accordingly.

Suitable C₂₋₆ alkenyl groups include vinyl, allyl and prop-1-en-2-yl.

Suitable C₃₋₇ cycloalkyl groups, which may comprise benzo-fused analogues thereof, include cyclopropyl, cyclobutyl, cyclopentyl, indanyl, cyclohexyl and cycloheptyl.

Suitable aryl groups include phenyl and naphthyl, preferably phenyl.

Suitable aryl(C₁₋₆)alkyl groups include benzyl, phenylethyl, phenylpropyl and naphthylmethyl.

Suitable heterocycloalkyl groups, which may comprise benzo-fused analogues thereof, include oxetanyl, azetidinyl, tetrahydrofuranyl, dihydrobenzofuranyl, dihydro-isobenzofuranyl, pyrrolidinyl, indolinyl, thiazolidinyl, imidazolidinyl, tetrahydropyranyl, chromanyl, piperidinyl, 1,2,3,4-tetrahydroquinolinyl, 1,2,3,4-tetrahydroisoquinolinyl, piperazinyl, 1,2,3,4-tetrahydroquinoxalinyl, homopiperazinyl, morpholinyl, benzoxazinyl and thiomorpholinyl.

Examples of suitable heterocycloalkenyl groups include oxazolinyl.

Suitable heteroaryl groups include furyl, benzofuryl, dibenzofuryl, thienyl, benzothienyl, dibenzothienyl, pyrrolyl, indolyl, pyrrolo[2,3-b]pyridinyl, pyrrolo[3,2-c]-pyridinyl, pyrazolyl, pyrazolo[1,5-c]pyridinyl, pyrazolo[3,4-d]pyrimidinyl, indazolyl, oxazolyl, benzoxazolyl, isoxazolyl, thiazolyl, benzothiazolyl, isothiazolyl, imidazolyl, imidazo[2,1-b]thiazolyl, benzimidazolyl, imidazo[1,2-c]pyridinyl, imidazo[1,5-c]-pyridinyl, imidazo[4,5-b]pyridinyl, purinyl, imidazo[1,2-c]pyrimidinyl, imidazo[1,2-c]-pyrazinyl, oxadiazolyl, benzoxadiazolyl, thiadiazolyl, benzothiadiazolyl, triazolyl, benzotriazolyl, [1,2,4]triazolo[4,3-c]pyridinyl, tetrazolyl, pyridinyl, quinolinyl, isoquinolinyl, naphthyridinyl, pyridazinyl, cinnolinyl, phthalazinyl, pyrimidinyl, quinazolinyl, pyrazinyl, quinoxalinyl, pteridinyl, triazinyl and chromenyl groups.

The term “halogen” as used herein is intended to include fluorine, chlorine, bromine and iodine atoms, typically fluorine, chlorine or bromine.

Where the compounds of formula (I) have one or more asymmetric centres, they may accordingly exist as enantiomers. Where the compounds of the invention possess two or more asymmetric centres, they may additionally exist as diastereomers. The invention is to be understood to extend to all such enantiomers and diastereomers, and to mixtures thereof in any proportion, including racemates. Formula (I) and the formulae depicted hereinafter are intended to represent all individual stereoisomers and all possible mixtures thereof, unless stated or shown otherwise. In addition, compounds of formula (I) may exist as tautomers, for example keto (CH₂C═O)↔enol (CH═CHOH) tautomers or amide (NHC═O)↔hydroxyimine (N═COH) tautomers. Formula (I) and the formulae depicted hereinafter are intended to represent all individual tautomers and all possible mixtures thereof, unless stated or shown otherwise.

It is to be understood that each individual atom present in formula (I), or in the formulae depicted hereinafter, may in fact be present in the form of any of its naturally occurring isotopes, with the most abundant isotope(s) being preferred. Thus, by way of example, each individual hydrogen atom present in formula (I), or in the formulae depicted hereinafter, may be present as a ¹H, ²H (deuterium) or ³H (tritium) atom, preferably ¹H. Similarly, by way of example, each individual carbon atom present in formula (I), or in the formulae depicted hereinafter, may be present as a ¹²C, ¹³C or ¹⁴C atom, preferably ¹²C.

In one embodiment, X represents N. In another embodiment, X represents CH.

Individual sub-classes of compounds in accordance with the present invention are represented by the compounds of formula (IA) and (IB):

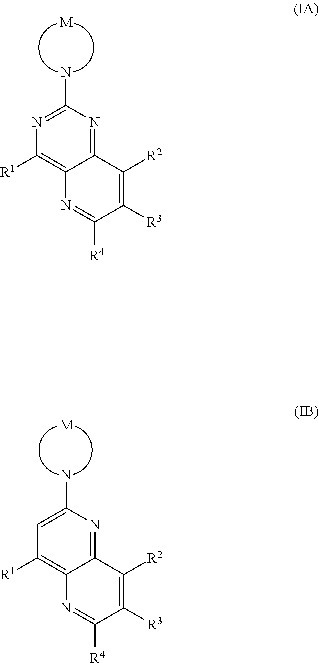

wherein M, R¹, R², R³ and R⁴ are as defined above.

In a first aspect, M represents the residue of an optionally substituted saturated four-, five-, six- or seven-membered monocyclic ring containing one nitrogen atom and 0, 1, 2 or 3 additional heteroatoms independently selected from N, O and S, but containing no more than one O or S atom.

In a first embodiment, M represents the residue of an optionally substituted saturated four-membered monocyclic ring. In a second embodiment, M represents the residue of an optionally substituted saturated five-membered monocyclic ring. In a third embodiment, M represents the residue of an optionally substituted saturated six-membered monocyclic ring. In a fourth embodiment, M represents the residue of an optionally substituted saturated seven-membered monocyclic ring.

In a first embodiment, the monocyclic ring of which M is the residue contains one nitrogen atom and no additional heteroatoms (i.e. it is an optionally substituted azetidin-1-yl, pyrrolidin-1-yl, piperidin-1-yl or azepan-1-yl ring). In a second embodiment, the monocyclic ring of which M is the residue contains one nitrogen atom and one additional heteroatom selected from N, O and S. In a third embodiment, the monocyclic ring of which M is the residue contains one nitrogen atom and two additional heteroatoms selected from N, O and S, of which not more than one is O or S. In a fourth embodiment, the monocyclic ring of which M is the residue contains one nitrogen atom and three additional heteroatoms selected from N, O and S, of which not more than one is O or S.

Typical values of the monocyclic ring of which M is the residue include azetidin-1-yl, pyrrolidin-1-yl, imidazolidin-1-yl, piperidin-1-yl, morpholin-4-yl, thiomorpholin-4-yl, piperazin-1-yl, azepan-1-yl and [1,4]diazepan-1-yl, any of which rings may be optionally substituted by one or more substituents.

Suitable values of the monocyclic ring of which M is the residue include azetidin-1-yl, morpholin-4-yl, piperazin-1-yl and azepan-1-yl, any of which rings may be optionally substituted by one or more substituents.

A particular value of the monocyclic ring of which M is the residue is optionally substituted piperazin-1-yl.

In a second aspect, M represents the residue of an optionally substituted saturated or unsaturated 5- to 10-membered fused bicyclic ring system containing one nitrogen atom and 0, 1, 2 or 3 additional heteroatoms independently selected from N, O and S, but containing no more than one O or S atom.

In a first embodiment, M represents the residue of an optionally substituted saturated or unsaturated five-membered fused bicyclic ring system. In a second embodiment, M represents the residue of an optionally substituted saturated or unsaturated six-membered fused bicyclic ring system. In a third embodiment, M represents the residue of an optionally substituted saturated or unsaturated seven-membered fused bicyclic ring system. In a fourth embodiment, M represents the residue of an optionally substituted saturated or unsaturated eight-membered fused bicyclic ring system. In a fifth embodiment, M represents the residue of an optionally substituted saturated or unsaturated nine-membered fused bicyclic ring system. In a sixth embodiment, M represents the residue of an optionally substituted saturated or unsaturated ten-membered fused bicyclic ring system.

In a first embodiment, the fused bicyclic ring system of which M is the residue is saturated. In a second embodiment, the fused bicyclic ring system of which M is the residue is unsaturated.

In a first embodiment, the fused bicyclic ring system of which M is the residue contains one nitrogen atom and no additional heteroatoms. In a second embodiment, the fused bicyclic ring system of which M is the residue contains one nitrogen atom and one additional heteroatom selected from N, O and S. In a third embodiment, the fused bicyclic ring system of which M is the residue contains one nitrogen atom and two additional heteroatoms selected from N, O and S, of which not more than one is O or S. In a fourth embodiment, the fused bicyclic ring system of which M is the residue contains one nitrogen atom and three additional heteroatoms selected from N, O and S, of which not more than one is O or S.

Typical values of the fused bicyclic ring system of which M is the residue include 1,2,3,4,6,7,8,8a-octahydropyrrolo[1,2-a]pyrazin-2-yl and 4,5,6,7-tetrahydropyrazolo[1,5-a]pyrazin-5-yl, either of which ring systems may be optionally substituted by one or more substituents.

Suitable values of the fused bicyclic ring system of which M is the residue include 1,2,3,4,6,7,8,8a-octahydropyrrolo[1,2-a]pyrazin-2-yl, which ring system may be optionally substituted by one or more substituents.

In a third aspect, M represents the residue of an optionally substituted saturated 5- to 9-membered bridged bicyclic ring system containing one nitrogen atom and 0, 1, 2 or 3 additional heteroatoms independently selected from N, O and S, but containing no more than one O or S atom.

In a first embodiment, M represents the residue of an optionally substituted saturated five-membered bridged bicyclic ring system. In a second embodiment, M represents the residue of an optionally substituted saturated six-membered bridged bicyclic ring system. In a third embodiment, M represents the residue of an optionally substituted saturated seven-membered bridged bicyclic ring system. In a fourth embodiment, M represents the residue of an optionally substituted saturated eight-membered bridged bicyclic ring system. In a fifth embodiment, M represents the residue of an optionally substituted saturated nine-membered bridged bicyclic ring system.

In a first embodiment, the bridged bicyclic ring system of which M is the residue contains one nitrogen atom and no additional heteroatoms. In a second embodiment, the bridged bicyclic ring system of which M is the residue contains one nitrogen atom and one additional heteroatom selected from N, O and S. In a third embodiment, the bridged bicyclic ring system of which M is the residue contains one nitrogen atom and two additional heteroatoms selected from N, O and S, of which not more than one is O or S. In a fourth embodiment, the bridged bicyclic ring system of which M is the residue contains one nitrogen atom and three additional heteroatoms selected from N, O and S, of which not more than one is O or S.

Typical values of the bridged bicyclic ring system of which M is the residue include 3-azabicyclo[3.1.0]hexan-3-yl, 2-oxa-5-azabicyclo[2.2.1]heptan-5-yl, 6-azabicyclo[3.2.0]heptan-6-yl, 3-azabicyclo[3.1.1]heptan-3-yl, 3-azabicyclo[4.1.0]heptan-3-yl, 2-oxa-5-azabicyclo[2.2.2]octan-5-yl, 3-azabicyclo[3.2.1]octan-3-yl, 8-azabicyclo-[3.2.1]octan-8-yl, 3-oxa-8-azabicyclo[3.2.1]octan-8-yl, 3,8-diazabicyclo[3.2.1]octan-3-yl, 3,8-diazabicyclo[3.2.1]octan-8-yl, 3,6-diazabicyclo[3.2.2]nonan-3-yl, 3,6-diazabicyclo-[3.2.2]nonan-6-yl, 3-oxa-7-azabicyclo[3.3.1]nonan-7-yl, 3,9-diazabicyclo[4.2.1]nonan-3-yl and 3,9-diazabicyclo[4.2.1]nonan-9-yl, any of which ring systems may be optionally substituted by one or more substituents.

In a fourth aspect, M represents the residue of an optionally substituted saturated 5- to 9-membered spirocyclic ring system containing one nitrogen atom and 0, 1, 2 or 3 additional heteroatoms independently selected from N, O and S, but containing no more than one O or S atom.

In a first embodiment, M represents the residue of an optionally substituted saturated five-membered spirocyclic ring system. In a second embodiment, M represents the residue of an optionally substituted saturated six-membered spirocyclic ring system. In a third embodiment, M represents the residue of an optionally substituted saturated seven-membered spirocyclic ring system. In a fourth embodiment, M represents the residue of an optionally substituted saturated eight-membered spirocyclic ring system. In a fifth embodiment, M represents the residue of an optionally substituted saturated nine-membered spirocyclic ring system.

In a first embodiment, the spirocyclic ring system of which M is the residue contains one nitrogen atom and no additional heteroatoms. In a second embodiment, the spirocyclic ring system of which M is the residue contains one nitrogen atom and one additional heteroatom selected from N, O and S. In a third embodiment, the spirocyclic ring system of which M is the residue contains one nitrogen atom and two additional heteroatoms selected from N, O and S, of which not more than one is O or S. In a fourth embodiment, the spirocyclic ring system of which M is the residue contains one nitrogen atom and three additional heteroatoms selected from N, O and S, of which not more than one is O or S.

Typical values of the spirocyclic ring system of which M is the residue include 5-azaspiro[2.3]hexan-5-yl, 5-azaspiro[2.4]heptan-5-yl, 2-azaspiro[3.3]heptan-2-yl, 2-oxa-6-azaspiro[3.3]heptan-6-yl, 2-oxa-6-azaspiro[3.4]octan-6-yl, 2-oxa-6-azaspiro[3.5]nonan-2-yl, 7-oxa-2-azaspiro[3.5]nonan-2-yl and 2-oxa-7-azaspiro[3.5]nonan-7-yl, any of which ring systems may be optionally substituted by one or more substituents.

Suitable values of the spirocyclic ring system of which M is the residue include 2-oxa-6-azaspiro[3.3]heptan-6-yl, which ring system may be optionally substituted by one or more substituents.

In a first embodiment, the cyclic moiety of which M is the residue is unsubstituted. In a second embodiment, the cyclic moiety of which M is the residue is substituted by one or more substituents. In one subset of that embodiment, the cyclic moiety of which M is the residue is monosubstituted. In another subset of that embodiment, the cyclic moiety of which M is the residue is disubstituted.

Typical examples of optional substituents on the cyclic moiety of which M is the residue include halogen, C₁₋₆ alkyl, benzyl, heteroaryl, C₁₋₆ alkoxy, difluoromethoxy, trifluoromethoxy, C₁₋₆ alkoxy(C₁₋₆)alkyl, C₁₋₆ alkylthio, C₁₋₆ alkylsulfonyl, hydroxy, hydroxy(C₁₋₆)alkyl, cyano, trifluoromethyl, oxo, C₂₋₆ alkylcarbonyl, hydroxy(C₁₋₆)alkyl-carbonyl, di(C₁₋₆)alkylamino(C₁₋₆)alkylcarbonyl, carboxy, carboxy(C₁₋₆)alkyl, C₂₋₆ alkoxycarbonyl, C₂₋₆ alkoxycarbonyl(C₁₋₆)alkyl, amino, amino(C₁₋₆)alkyl, C₁₋₆ alkylamino, di(C₁₋₆)alkylamino, phenylamino, pyridinylamino, C₂₋₆ alkylcarbonylamino, hydroxy-(C₁₋₆)alkylcarbonylamino, (C₃₋₇)cycloalkylcarbonylamino, C₂₋₆ alkoxycarbonylamino, C₁₋₆ alkylsulfonylamino, aminocarbonyl, C₁₋₆ alkylaminocarbonyl, di(C₁₋₆)alkylamino-carbonyl, aminocarbonyl(C₁₋₆)alkyl, (C₁₋₆)alkylaminocarbonyl(C₁₋₆)alkyl, di(C₁₋₆)alkyl-aminocarbonyl(C₁₋₆)alkyl, (C₁₋₆ alkoxy)(C₁₋₆ alkyl)phenylaminocarbonyl, (C₁₋₆ alkoxy)-(C₁₋₆ alkyl)pyridinylaminocarbonyl, [di(C₁₋₆)alkylamino](C₁₋₆ alkyl)pyridinylamino-carbonyl and (dihaloazetidinyl)(C₁₋₆ alkyl)pyridinylaminocarbonyl.

Suitable examples of optional substituents on the cyclic moiety of which M is the residue include C₁₋₆ alkyl, C₂₋₆ alkylcarbonyl, C₂₋₆ alkoxycarbonyl, (C₁₋₆ alkoxy)(C₁₋₆ alkyl)phenylaminocarbonyl, (C₁₋₆ alkoxy)(C₁₋₆ alkyl)pyridinylaminocarbonyl, [di(C₁₋₆)-alkylamine](C₁₋₆ alkyl)pyridinylaminocarbonyl and (dihaloazetidinyl)(C₁₋₆ alkyl)-pyridinylaminocarbonyl.

Typical examples of specific substituents on the cyclic moiety of which M is the residue include fluoro, chloro, bromo, methyl, ethyl, propyl, isopropyl, benzyl, pyridinyl, pyrazinyl, methoxy, isopropoxy, difluoromethoxy, trifluoromethoxy, methoxymethyl, methylthio, ethylthio, methylsulfonyl, hydroxy, hydroxymethyl, hydroxyethyl, cyano, trifluoromethyl, oxo, acetyl, ethylcarbonyl, tert-butylcarbonyl, hydroxyacetyl, dimethyl-aminoacetyl, carboxy, carboxymethyl, methoxycarbonyl, ethoxycarbonyl, tert-butoxy-carbonyl, methoxycarbonylmethyl, ethoxycarbonylmethyl, amino, aminomethyl, methyl-amino, ethylamino, dimethylamino, phenylamino, pyridinylamino, acetylamino, hydroxyacetylamino, cyclopropylcarbonylamino, tert-butoxycarbonylamino, methyl-sulfonylamino, aminocarbonyl, methylaminocarbonyl, dimethylaminocarbonyl, amino-carbonylmethyl, methylaminocarbonylmethyl, dimethylaminocarbonylmethyl, (methoxy)-(methyl)phenylaminocarbonyl, (methoxy)(methyl)pyridinylaminocarbonyl, (dimethyl-amino)(methyl)pyridinylaminocarbonyl and (difluoroazetidinyl)(methyl)pyridinylamino-carbonyl.

Suitable examples of specific substituents on the cyclic moiety of which M is the residue include methyl, acetyl, ethoxycarbonyl, (methoxy)(methyl)phenylaminocarbonyl, (methoxy)(methyl)pyridinylaminocarbonyl, (dimethylamino)(methyl)pyridinylamino-carbonyl and (difluoroazetidinyl)(methyl)pyridinylaminocarbonyl.

Typical values of the cyclic moiety of which M is the residue include 3,3-difluoro-azetidin-1-yl, pyrrolidin-1-yl, 3-hydroxypyrrolidin-1-yl, 3-(acetylamino)pyrrolidin-1-yl, 3-(hydroxyacetylamino)pyrrolidin-1-yl, imidazolidin-1-yl, 4-hydroxypiperidin-1-yl, 4-carboxypiperidin-1-yl, 4-(acetylamino)piperidin-1-yl, 4-(methylsulfonylamino)piperidin-1-yl, 4-(aminocarbonyl)piperidin-1-yl, 4-(methylaminocarbonyl)piperidin-1-yl, morpholin-4-yl, 3-methylmorpholin-4-yl, thiomorpholin-4-yl, 1,1-dioxothiomorpholin-4-yl, piperazin-1-yl, 4-methylpiperazin-1-yl, 4-ethylpiperazin-1-yl, 4-propylpiperazin-1-yl, 4-isopropylpiperazin-1-yl, 4-benzylpiperazin-1-yl, 4-(pyridin-2-yl)piperazin-1-yl, 4-(pyrazin-2-yl)piperazin-1-yl, 4-(methylsulfonyl)piperazin-1-yl, 4-(2-hydroxyethyl)-piperazin-1-yl, 3-oxopiperazin-1-yl, 4-methyl-3-oxopiperazin-1-yl, 4-acetylpiperazin-1-yl, 4-(ethylcarbonyl)piperazin-1-yl, 4-(tert-butylcarbonyl)piperazin-1-yl, 4-(hydroxyacetyl)piperazin-1-yl, 4-(dimethylaminoacetyl)piperazin-1-yl, 4-(carboxy-methyl)piperazin-1-yl, 4-(methoxycarbonyl)piperazin-1-yl, 4-(ethoxycarbonyl)piperazin-1-yl, 4-(ethoxycarbonylmethyl)piperazin-1-yl, 4-(aminocarbonyl)piperazin-1-yl, 4-(aminocarbonylmethyl)piperazin-1-yl, 4-(methylaminocarbonylmethyl)piperazin-1-yl, 4-(dimethylaminocarbonylmethyl)piperazin-1-yl, 4-[(4-methoxy-2-methylphenyl)amino-carbonyl]piperazin-1-yl, 4-[(4-methoxy-2-methylphenyl)aminocarbonyl]-2-methyl-piperazin-1-yl, 4-[(6-methoxy-2-methylpyridin-3-yl)aminocarbonyl]-2-methylpiperazin-1-yl, 4-{[6-(dimethylamino)-2-methylpyridin-3-yl]aminocarbonyl}-2-methylpiperazin-1-yl, 4-{[6-(3,3-difluoroazetidin-1-yl)-2-methylpyridin-3-yl]aminocarbonyl}-2-methyl-piperazin-1-yl, azepan-1-yl, 5-oxo-[1,4]diazepan-1-yl, 6-oxo-1,3,4,7,8,8a-hexahydro-pyrrolo[1,2-c]pyrazin-2-yl, 4,5,6,7-tetrahydropyrazolo[1,5-c]pyrazin-5-yl and 2-oxa-6-azaspiro[3.3]heptan-6-yl.

Suitable values of the cyclic moiety of which M is the residue include 4-acetyl-piperazin-1-yl, 4-(ethoxycarbonyl)piperazin-1-yl, 4-[(4-methoxy-2-methylphenyl)amino-carbonyl]piperazin-1-yl, 4-[(4-methoxy-2-methylphenyl)aminocarbonyl]-2-methyl-piperazin-1-yl, 4-[(6-methoxy-2-methylpyridin-3-yl)aminocarbonyl]-2-methylpiperazin-1-yl, 4-{[6-(dimethylamino)-2-methylpyridin-3-yl]aminocarbonyl}-2-methylpiperazin-1-yl and 4-{[6-(3,3-difluoroazetidin-1-yl)-2-methylpyridin-3-yl]aminocarbonyl}-2-methyl-piperazin-1-yl.

Suitably, R¹ represents hydrogen, halogen, cyano, nitro, hydroxy, trifluoromethyl, trifluoromethoxy, —OR^{a}, —SR^{a}, —SO₂R^{a}, —NR^{b}R^{c}, —CH₂NR^{b}R^{c}, —NR^{c}COR^{d}, —CH₂NR^{c}COR^{d}, —NR^{c}CO₂R^{d}, —NHCONR^{b}R^{c}, —NR^{c}SO₂R^{e}, —NHSO₂NR^{b}R^{c}, —COR^{d}, —CO₂R^{d}, —CONR^{b}R^{c}, —CON(OR^{a})R^{b} or —SO₂NR^{b}R^{c}; or R¹ represents C₁₋₆ alkyl, aryl or heteroaryl, any of which groups may be optionally substituted by one or more substituents.

Typically, R¹ represents hydrogen, —OR^{a}, —SR^{a}, —SO₂R^{a}, —NR^{b}R^{c} or —NR^{c}COR^{d}; or R¹ represents C₁₋₆ alkyl, which group may be optionally substituted by one or more substituents.

Typical values of R¹ include hydrogen, —OR^{a}, —SR^{a}, —SO₂R^{a} and —NR^{b}R^{c}.

Suitable values of R¹ include hydrogen and —NR^{b}R^{c}.

In a first embodiment, R¹ represents hydrogen. In a second embodiment, R¹ represents cyano. In a third embodiment, R¹ represents —OR^{a}. In a fourth embodiment, R¹ represents —SR^{a}. In a fifth embodiment, R¹ represents —SO₂R^{a}. In a sixth embodiment, R¹ represents —NR^{b}R^{c}. In a seventh embodiment, R¹ represents —NR^{c}COR^{d}. In an eighth embodiment, R¹ represents optionally substituted C₁₋₆ alkyl. In one aspect of that embodiment, R¹ represents optionally substituted methyl.

Examples of typical substituents on R¹ include one or more substituents independently selected from halogen, cyano, nitro, C₁₋₆ alkyl, trifluoromethyl, aryl(C₁₋₆)alkyl, hydroxy, C₁₋₆ alkoxy, difluoromethoxy, trifluoromethoxy, aryloxy, C₁₋₄ alkylenedioxy, C₁₋₆ alkoxy(C₁₋₆)alkyl, C₁₋₆ alkylthio, C₁₋₆ alkylsulfonyl, oxo, amino, C₁₋₆ alkylamino, di(C₁₋₆)alkylamino, C₂₋₆ alkylcarbonylamino, C₂₋₆ alkoxycarbonylamino, aryl(C₁₋₆)alkoxycarbonylamino, C₁₋₆ alkylaminocarbonylamino, arylaminocarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylaminocarbonyl, aminosulfonyl, C₁₋₆ alkylaminosulfonyl and di(C₁₋₆)alkylaminosulfonyl.

Specific examples of typical substituents on R¹ include one or more substituents independently selected from fluoro, chloro, bromo, cyano, nitro, methyl, ethyl, tert-butyl, trifluoromethyl, benzyl, hydroxy, methoxy, difluoromethoxy, trifluoromethoxy, phenoxy, methylenedioxy, ethylenedioxy, methoxymethyl, methylthio, methylsulfonyl, oxo, amino, methylamino, dimethylamino, acetylamino, methoxycarbonylamino, ethoxycarbonyl-amino, benzyloxycarbonylamino, ethylaminocarbonylamino, butylaminocarbonylamino, phenylaminocarbonylamino, methylsulfonylamino, formyl, acetyl, carboxy, methoxycarbonyl, aminocarbonyl, methylaminocarbonyl, dimethylaminocarbonyl, aminosulfonyl, methylaminosulfonyl and dimethylaminosulfonyl.

Generally, R² represents hydrogen, cyano, hydroxy, trifluoromethyl, —NR^{c}O₂R^{d}, —COR^{d}, —CO₂R^{d}, —CONR^{b} R^{c} or —CON(OR^{a})R^{b}; or R² represents C₁₋₆ alkyl, C₃₋₇ cycloalkyl, aryl, C₃₋₇ heterocycloalkyl, C₃₋₇ heterocycloalkenyl or heteroaryl, any of which groups may be optionally substituted by one or more substituents.

Typically, R² represents hydrogen; or R² represents aryl, C₃₋₇ heterocycloalkyl or heteroaryl, any of which groups may be optionally substituted by one or more substituents.

Suitably, R² represents aryl or heteroaryl, either of which groups may be optionally substituted by one or more substituents.

Appositely, R² represents hydrogen; or R² represents aryl, which group may be optionally substituted by one or more substituents.

In a first embodiment, R² represents hydrogen. In a second embodiment, R² represents cyano. In a third embodiment, R² represents hydroxy. In a fourth embodiment, R² represents trifluoromethyl. In a fifth embodiment, R² represents —NR^{c}O₂R^{d}. In a sixth embodiment, R² represents —COR^{d}. In a seventh embodiment, R² represents —CO₂R^{d}. In an eighth embodiment, R² represents —CONR^{b}R^{c}. In a ninth embodiment, R² represents —CON(OR^{a})R^{b}. In a tenth embodiment, R² represents optionally substituted C₁₋₆ alkyl. In a first aspect of that embodiment, R² represents unsubstituted C₁₋₆ alkyl. In a second aspect of that embodiment, R² represents monosubstituted C₁₋₆ alkyl. In a third aspect of that embodiment, R² represents disubstituted C₁₋₆ alkyl. In an eleventh embodiment, R² represents optionally substituted C₃₋₇ cycloalkyl. In a first aspect of that embodiment, R² represents unsubstituted C₃₋₇ cycloalkyl. In a second aspect of that embodiment, R² represents monosubstituted C₃₋₇ cycloalkyl. In a third aspect of that embodiment, R² represents disubstituted C₃₋₇ cycloalkyl. In a twelfth embodiment, R² represents optionally substituted aryl. In a first aspect of that embodiment, R² represents unsubstituted aryl. In a second aspect of that embodiment, R² represents monosubstituted aryl. In a third aspect of that embodiment, R² represents disubstituted aryl. In a thirteenth embodiment, R² represents optionally substituted C₃₋₇ heterocycloalkyl. In a first aspect of that embodiment, R² represents unsubstituted C₃₋₇ heterocycloalkyl. In a second aspect of that embodiment, R² represents monosubstituted C₃₋₇ heterocycloalkyl. In a third aspect of that embodiment, R² represents disubstituted C₃₋₇ heterocycloalkyl. In a fourteenth embodiment, R² represents optionally substituted C₃₋₇ heterocycloalkenyl. In a first aspect of that embodiment, R² represents unsubstituted C₃₋₇ heterocycloalkenyl. In a second aspect of that embodiment, R² represents monosubstituted C₃₋₇ heterocycloalkenyl. In a third aspect of that embodiment, R² represents disubstituted C₃₋₇ heterocycloalkenyl. In a fifteenth embodiment, R² represents optionally substituted heteroaryl. In a first aspect of that embodiment, R² represents unsubstituted heteroaryl. In a second aspect of that embodiment, R² represents monosubstituted heteroaryl. In a third aspect of that embodiment, R² represents disubstituted heteroaryl.

Where R² represents optionally substituted C₁₋₆ alkyl, suitable values include methyl, ethyl, n-propyl, isopropyl, n-butyl, isobutyl and tert-butyl, any of which groups may be optionally substituted by one or more substituents. Selected values include methyl, hydroxymethyl, chloropropyl and isobutyl. Particular values include methyl and isobutyl, especially methyl.

Where R² represents optionally substituted C₃₋₇ cycloalkyl, a suitable value is cyclohexyl, optionally substituted by one or more substituents.

Where R² represents optionally substituted aryl, a suitable value is phenyl, optionally substituted by one or more substituents.

Where R² represents optionally substituted C₃₋₇ heterocycloalkyl, typical values include azetidinyl, dihydroisobenzofuranyl, pyrrolidinyl, indolinyl, piperidinyl, piperazinyl, morpholinyl and thiomorpholinyl, any of which groups may be optionally substituted by one or more substituents.

Where R² represents optionally substituted C₃₋₇ heterocycloalkenyl, a typical value is oxazolinyl, optionally substituted by one or more substituents. Suitable values include oxazolinyl, methyloxazolinyl, isopropyloxazolinyl and dimethyloxazolinyl.

Where R² represents optionally substituted heteroaryl, typical values include furyl, thienyl, pyrrolyl, pyrazolyl, indazolyl, oxazolyl, isoxazolyl, thiazolyl, isothiazolyl, imidazolyl, imidazo[1,5-a]pyridinyl, oxadiazolyl, benzoxadiazolyl, thiadiazolyl, triazolyl, [1,2,4]triazolo[4,3-a]pyridinyl, tetrazolyl, pyridinyl, pyridazinyl, pyrimidinyl, pyrazinyl and triazinyl, any of which groups may be optionally substituted by one or more substituents.

In a typical embodiment, R² represents hydrogen; or R² represents phenyl, dihydroisobenzofuranyl, indolinyl, indazolyl, imidazo[1,5-a]pyridinyl, benzoxadiazolyl, [1,2,4]triazolo[4,3-a]pyridinyl or pyridinyl, any of which groups may be optionally substituted by one or more substituents.

In a suitable embodiment, R² represents hydrogen; or R² represents phenyl, which group may be optionally substituted by one or more substituents.

Typical examples of optional substituents on R² include one or more substituents independently selected from halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, oxo, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylamino-carbonyl, aminosulfonyl, C₁₋₆ alkylaminosulfonyl and di(C₁₋₆)alkylaminosulfonyl.

Suitable examples of optional substituents on R² include one or more substituents independently selected from C₁₋₆ alkoxy.

Typical examples of specific substituents on R² include one or more substituents independently selected from fluoro, chloro, bromo, cyano, nitro, methyl, ethyl, isopropyl, tert-butyl, trifluoromethyl, hydroxy, methoxy, isopropoxy, difluoromethoxy, trifluoro-methoxy, methylthio, methylsulfinyl, methylsulfonyl, oxo, amino, methylamino, dimethylamino, acetylamino, methoxycarbonylamino, methylsulfonylamino, formyl, acetyl, carboxy, methoxycarbonyl, aminocarbonyl, methylaminocarbonyl, dimethyl-aminocarbonyl, aminosulfonyl, methylaminosulfonyl and dimethylaminosulfonyl.

Suitable examples of specific substituents on R² include one or more substituents independently selected from methoxy.

Typical values of R² include hydrogen, cyano, hydroxy, trifluoromethyl, —NR²O₂R^{d}, —COR^{d}, —CO₂R^{d}, —CONR^{b}R^{c}, —CON(OR^{a})R^{b}, methyl, hydroxymethyl, chloro-propyl, isobutyl, cyclohexyl, phenyl, fluorophenyl, chlorophenyl, methoxyphenyl, (fluoro)(methoxy)phenyl, dimethoxyphenyl, (difluoromethoxy)(methoxy)phenyl, (methoxy)(methylsulfonyl)phenyl, (chloro)(methylaminocarbonyl)phenyl, oxo-3H-isobenzofuranyl, (methyl)(oxo)indolinyl, oxazolinyl, methyloxazolinyl, isopropyl-oxazolinyl, dimethyloxazolinyl, methylindazolyl, dimethylindazolyl, dimethylimidazo-[1,5-a]pyridinyl, methyloxadiazolyl, isopropyloxadiazolyl, tert-butyloxadiazolyl, benzoxadiazolyl, methyl[1,2,4]triazolo[4,3-a]pyridinyl, pyridinyl and dimethoxy-pyridinyl.

Suitable values of R² include hydrogen and dimethoxyphenyl.

Generally, R³ represents hydrogen, cyano, hydroxy, trifluoromethyl, —NR^{c}O₂R^{d}, —COR^{d}, —CO₂R^{d}, —CONR^{b}R^{c} or —CON(OR^{a})R^{b}; or R³ represents C₁₋₆ alkyl, C₃₋₇ cycloalkyl, aryl, C₃₋₇ heterocycloalkyl, C₃₋₇ heterocycloalkenyl or heteroaryl, any of which groups may be optionally substituted by one or more substituents.

Typically, R³ represents hydrogen; or R³ represents aryl, C₃₋₇ heterocycloalkyl or heteroaryl, any of which groups may be optionally substituted by one or more substituents.

Suitably, R³ represents aryl or heteroaryl, either of which groups may be optionally substituted by one or more substituents.

In a first embodiment, R³ represents hydrogen. In a second embodiment, R³ represents cyano. In a third embodiment, R³ represents hydroxy. In a fourth embodiment, R³ represents trifluoromethyl. In a fifth embodiment, R³ represents —NR^{c}O₂R^{d}. In a sixth embodiment, R³ represents —COR^{d}. In a seventh embodiment, R³ represents —CO₂R^{d}. In an eighth embodiment, R³ represents —CONR^{b}R^{c}. In a ninth embodiment, R³ represents —CON(OR^{a})R^{b}. In a tenth embodiment, R³ represents optionally substituted C₁₋₆ alkyl. In a first aspect of that embodiment, R³ represents unsubstituted C₁₋₆ alkyl. In a second aspect of that embodiment, R³ represents monosubstituted C₁₋₆ alkyl. In a third aspect of that embodiment, R³ represents disubstituted C₁₋₆ alkyl. In an eleventh embodiment, R³ represents optionally substituted C₃₋₇ cycloalkyl. In a first aspect of that embodiment, R³ represents unsubstituted C₃₋₇ cycloalkyl. In a second aspect of that embodiment, R³ represents monosubstituted C₃₋₇ cycloalkyl. In a third aspect of that embodiment, R³ represents disubstituted C₃₋₇ cycloalkyl. In a twelfth embodiment, R³ represents optionally substituted aryl. In a first aspect of that embodiment, R³ represents unsubstituted aryl. In a second aspect of that embodiment, R³ represents monosubstituted aryl. In a third aspect of that embodiment, R³ represents disubstituted aryl. In a thirteenth embodiment, R³ represents optionally substituted C₃₋₇ heterocycloalkyl. In a first aspect of that embodiment, R³ represents unsubstituted C₃₋₇ heterocycloalkyl. In a second aspect of that embodiment, R³ represents monosubstituted C₃₋₇ heterocycloalkyl. In a third aspect of that embodiment, R³ represents disubstituted C₃₋₇ heterocycloalkyl. In a fourteenth embodiment, R³ represents optionally substituted C₃₋₇ heterocycloalkenyl. In a first aspect of that embodiment, R³ represents unsubstituted C₃₋₇ heterocycloalkenyl. In a second aspect of that embodiment, R³ represents monosubstituted C₃₋₇ heterocycloalkenyl. In a third aspect of that embodiment, R³ represents disubstituted C₃₋₇ heterocycloalkenyl. In a fifteenth embodiment, R³ represents optionally substituted heteroaryl. In a first aspect of that embodiment, R³ represents unsubstituted heteroaryl. In a second aspect of that embodiment, R³ represents monosubstituted heteroaryl. In a third aspect of that embodiment, R³ represents disubstituted heteroaryl.

Where R³ represents optionally substituted C₁₋₆ alkyl, suitable values include methyl, ethyl, n-propyl, isopropyl, n-butyl, isobutyl and tert-butyl, any of which groups may be optionally substituted by one or more substituents. Selected values include methyl, hydroxymethyl, chloropropyl and isobutyl. Particular values include methyl and isobutyl, especially methyl.

Where R³ represents optionally substituted C₃₋₇ cycloalkyl, a suitable value is cyclohexyl, optionally substituted by one or more substituents.

Where R³ represents optionally substituted aryl, a suitable value is phenyl, optionally substituted by one or more substituents.

Where R³ represents optionally substituted C₃₋₇ heterocycloalkyl, typical values include azetidinyl, dihydroisobenzofuranyl, pyrrolidinyl, indolinyl, piperidinyl, piperazinyl, morpholinyl and thiomorpholinyl, any of which groups may be optionally substituted by one or more substituents.

Where R³ represents optionally substituted C₃₋₇ heterocycloalkenyl, a typical value is oxazolinyl, optionally substituted by one or more substituents. Suitable values include oxazolinyl, methyloxazolinyl, isopropyloxazolinyl and dimethyloxazolinyl.

Where R³ represents optionally substituted heteroaryl, typical values include furyl, thienyl, pyrrolyl, pyrazolyl, indazolyl, oxazolyl, isoxazolyl, thiazolyl, isothiazolyl, imidazolyl, imidazo[1,5-c]pyridinyl, oxadiazolyl, benzoxadiazolyl, thiadiazolyl, triazolyl, [1,2,4]triazolo[4,3-a]pyridinyl, tetrazolyl, pyridinyl, pyridazinyl, pyrimidinyl, pyrazinyl and triazinyl, any of which groups may be optionally substituted by one or more substituents.

In a typical embodiment, R³ represents hydrogen, phenyl, dihydroisobenzofuranyl, indolinyl, indazolyl, imidazo[1,5-a]pyridinyl, benzoxadiazolyl, [1,2,4]triazolo[4,3-a]-pyridinyl or pyridinyl, any of which groups may be optionally substituted by one or more substituents.

Typical examples of optional substituents on R³ include one or more substituents independently selected from halogen, cyano, nitro, C₁₋₆ alkyl, trifluoromethyl, hydroxy, C₁₋₆ alkoxy, difluoromethoxy, trifluoromethoxy, C₁₋₆ alkylthio, C₁₋₆ alkylsulfinyl, C₁₋₆ alkylsulfonyl, oxo, amino, C₁₋₆ alkylamino, di(C₁₋₆)alkylamino, C₂₋₆ alkylcarbonylamino, C₂₋₆ alkoxycarbonylamino, C₁₋₆ alkylsulfonylamino, formyl, C₂₋₆ alkylcarbonyl, carboxy, C₂₋₆ alkoxycarbonyl, aminocarbonyl, C₁₋₆ alkylaminocarbonyl, di(C₁₋₆)alkylamino-carbonyl, aminosulfonyl, C₁₋₆ alkylaminosulfonyl and di(C₁₋₆)alkylaminosulfonyl.

Typical examples of specific substituents on R³ include one or more substituents independently selected from fluoro, chloro, bromo, cyano, nitro, methyl, ethyl, isopropyl, tert-butyl, trifluoromethyl, hydroxy, methoxy, isopropoxy, difluoromethoxy, trifluoro-methoxy, methylthio, methylsulfinyl, methylsulfonyl, oxo, amino, methylamino, dimethylamino, acetylamino, methoxycarbonylamino, methylsulfonylamino, formyl, acetyl, carboxy, methoxycarbonyl, aminocarbonyl, methylaminocarbonyl, dimethyl-aminocarbonyl, aminosulfonyl, methylaminosulfonyl and dimethylaminosulfonyl.

Typical values of R³ include hydrogen, cyano, hydroxy, trifluoromethyl, —NR^{c}O₂R^{d}, —COR^{d}, —CO₂R^{d}, —CONR^{b}R^{c}, —CON(OR^{a})R^{b}, methyl, hydroxymethyl, chloro-propyl, isobutyl, cyclohexyl, phenyl, fluorophenyl, chlorophenyl, methoxyphenyl, (fluoro)(methoxy)phenyl, dimethoxyphenyl, (difluoromethoxy)(methoxy)phenyl, (methoxy)(methylsulfonyl)phenyl, (chloro)(methylaminocarbonyl)phenyl, oxo-3H-isobenzofuranyl, (methyl)(oxo)indolinyl, oxazolinyl, methyloxazolinyl, isopropyl-oxazolinyl, dimethyloxazolinyl, methylindazolyl, dimethylindazolyl, dimethylimidazo-[1,5-c]pyridinyl, methyloxadiazolyl, isopropyloxadiazolyl, tert-butyloxadiazolyl, benzoxadiazolyl, methyl[1,2,4]triazolo[4,3-a]pyridinyl, pyridinyl and dimethoxy-pyridinyl.

Typically, R⁴ represents hydrogen or C₁₋₆ alkyl.

In a first embodiment, R⁴ represents hydrogen. In a second embodiment, R⁴ represents halogen, especially fluoro or chloro. In a first aspect of that embodiment, R⁴ represents fluoro. In a second aspect of that embodiment, R⁴ represents chloro. In a third embodiment, R⁴ represents cyano. In a fourth embodiment, R⁴ represents trifluoromethyl. In a fifth embodiment, R⁴ represents C₁₋₆ alkyl, especially methyl.

Typical values of R⁴ include hydrogen, chloro, cyano, trifluoromethyl and methyl.

Suitable values of R⁴ include hydrogen and methyl.

Typical examples of suitable substituents on R^{a}, R^{b}, R^{c}, R^{d} or R^{e}, or on the heterocyclic moiety —NR^{b}R^{c}, include halogen, C₁₋₆ alkyl, C₁₋₆ alkoxy, difluoromethoxy, trifluoromethoxy, C₁₋₆ alkoxy(C₁₋₆)alkyl, C₁₋₆ alkylthio, C₁₋₆ alkylsulfinyl, C₁₋₆ alkylsulfonyl, C₁₋₆ alkylsulfonimidoyl, N,S-di(C₁₋₆)alkylsulfonimidoyl, hydroxy, hydroxy(C₁₋₆)alkyl, amino(C₁₋₆)alkyl, cyano, trifluoromethyl, oxo, C₂₋₆ alkylcarbonyl, carboxy, C₂₋₆ alkoxycarbonyl, C₂₋₆ alkylcarbonyloxy, amino, C₁₋₆ alkylamino, di-(C₁₋₆)alkylamino, phenylamino, pyridinylamino, C₂₋₆ alkylcarbonylamino, C₂₋₆ alkylcarbonylamino(C₁₋₆)alkyl, C₂₋₆ alkoxycarbonylamino, C₁₋₆ alkylsulfonylamino, aminocarbonyl, C₁₋₆ alkylaminocarbonyl and di(C₁₋₆)alkylaminocarbonyl.

Typical examples of specific substituents on R^{a}, R^{b}, R^{c}, R^{d} or R^{e}, or on the heterocyclic moiety —NR^{b}R^{c}, include fluoro, chloro, bromo, methyl, ethyl, isopropyl, methoxy, isopropoxy, difluoromethoxy, trifluoromethoxy, methoxymethyl, methylthio, ethylthio, methylsulfinyl, methylsulfonyl, methylsulfonimidoyl, N,S-dimethyl-sulfonimidoyl, hydroxy, hydroxymethyl, hydroxyethyl, aminomethyl, cyano, trifluoro-methyl, oxo, acetyl, carboxy, methoxycarbonyl, ethoxycarbonyl, tert-butoxycarbonyl, acetoxy, amino, methylamino, ethylamino, dimethylamino, phenylamino, pyridinylamino, acetylamino, acetylaminomethyl, tert-butoxycarbonylamino, methylsulfonylamino, aminocarbonyl, methylaminocarbonyl and dimethylaminocarbonyl.

Typically, R^{a} represents hydrogen; or R^{a} represents C₁₋₆ alkyl, aryl(C₁₋₆)alkyl or heteroaryl(C₁₋₆)alkyl, any of which groups may be optionally substituted by one or more substituents.

Suitably, R^{a} represents C₁₋₆ alkyl, aryl(C₁₋₆)alkyl or heteroaryl(C₁₋₆)alkyl, any of which groups may be optionally substituted by one or more substituents.

Apposite values of R^{a} include hydrogen; and methyl, ethyl, benzyl or isoindolyl-propyl, any of which groups may be optionally substituted by one or more substituents.

Selected values of R^{a} include methyl, ethyl, benzyl and isoindolylpropyl, any of which groups may be optionally substituted by one or more substituents.

Selected examples of suitable substituents on R^{a} include C₁₋₆ alkoxy and oxo.

Selected examples of specific substituents on R^{a} include methoxy and oxo.

In one embodiment, R^{a} represents hydrogen. In another embodiment, R^{a} represents optionally substituted C₁₋₆ alkyl. In one aspect of that embodiment, R^{a} ideally represents unsubstituted C₁₋₆ alkyl, especially methyl. In another aspect of that embodiment, R^{a} ideally represents substituted C₁₋₆ alkyl, e.g. methoxyethyl. In another embodiment, R^{a} represents optionally substituted aryl. In one aspect of that embodiment, R^{a} represents unsubstituted aryl, especially phenyl. In another aspect of that embodiment, R^{a} represents monosubstituted aryl, especially methylphenyl. In another embodiment, R^{a} represents optionally substituted aryl(C₁₋₆)alkyl, ideally unsubstituted aryl(C₁₋₆)alkyl, especially benzyl. In a further embodiment, R^{a} represents optionally substituted heteroaryl. In a further embodiment, R^{a} represents optionally substituted heteroaryl(C₁₋₆)alkyl, e.g. dioxoisoindolylpropyl.

Specific values of R^{a} include methyl, methoxyethyl, benzyl and dioxoisoindolyl-propyl.

Appositely, R^{a} represents hydrogen or C₁₋₆ alkyl.

Individual values of R^{a} include hydrogen and methyl.

In a typical aspect, R^{b} represents hydrogen or trifluoromethyl; or R^{b} represents C₁₋₆ alkyl, C₃₋₇ cycloalkyl, C₃₋₇ cycloalkyl(C₁₋₆)alkyl, aryl, aryl(C₁₋₆)alkyl, C₃₋₇ hetero-cycloalkyl, C₃₋₇ heterocycloalkyl(C₁₋₆)alkyl, heteroaryl or heteroaryl(C₁₋₆)alkyl, any of which groups may be optionally substituted by one or more substituents.

In a suitable aspect, R^{b} represents hydrogen; or R^{b} represents aryl(C₁₋₆)alkyl or heteroaryl(C₁₋₆)alkyl, either of which groups may be optionally substituted by one or more substituents.

Illustratively, R^{b} represents hydrogen or trifluoromethyl; or R^{b} represents methyl, ethyl, n-propyl, isopropyl, n-butyl, 2-methylpropyl, tert-butyl, pentyl, hexyl, cyclopropyl, cyclobutyl, cyclopentyl, cyclohexyl, cyclopropylmethyl, cyclobutylmethyl, cyclopentyl-methyl, cyclohexylmethyl, phenyl, benzyl, phenylethyl, azetidinyl, tetrahydrofuryl, tetrahydrothienyl, pyrrolidinyl, piperidinyl, homopiperidinyl, morpholinyl, azetidinylmethyl, tetrahydrofurylmethyl, pyrrolidinylmethyl, pyrrolidinylethyl, pyrrolidinylpropyl, thiazolidinylmethyl, imidazolidinylethyl, piperidinylmethyl, piperidinylethyl, tetrahydroquinolinylmethyl, piperazinylpropyl, morpholinylmethyl, morpholinylethyl, morpholinylpropyl, pyridinyl, indolylmethyl, isoxazolylmethyl, thiazolylmethyl, pyrazolylmethyl, pyrazolylethyl, imidazolylmethyl, imidazolylethyl, benzimidazolylmethyl, oxadiazolylmethyl, triazolylmethyl, pyridinylmethyl or pyridinylethyl, any of which groups may be optionally substituted by one or more substituents.

Typical examples of optional substituents on R^{b} include C₁₋₆ alkyl, C₁₋₆ alkoxy, C₁₋₆ alkylthio, C₁₋₆ alkylsulfinyl, C₁₋₆ alkylsulfonyl, C₁₋₆ alkylsulfonimidoyl, N,S-di-(C₁₋₆)alkylsulfonimidoyl, hydroxy, cyano, C₂₋₆ alkoxycarbonyl, di(C₁₋₆)alkylamino and C₂₋₆ alkoxycarbonylamino.

Typical examples of specific substituents on R^{b} include methyl, methoxy, methylthio, methylsulfinyl, methylsulfonyl, methylsulfonimidoyl, N,S-dimethyl-sulfonimidoyl, hydroxy, cyano, tert-butoxycarbonyl, dimethylamino and tert-butoxycarbonylamino.

Typical values of R^{b} include hydrogen, methyl, methoxyethyl, methylthioethyl, methylsulfinylethyl, methylsulfonylethyl, hydroxyethyl, cyanoethyl, dimethylaminoethyl, tert-butoxycarbonylaminoethyl, dihydroxypropyl, benzyl, methylsulfonylbenzyl, methyl-sulfonimidoylbenzyl, N,S-dimethylsulfonimidoylbenzyl, pyrrolidinyl, tert-butoxycarbonyl-pyrrolidinyl, morpholinylpropyl, methylisoxazolylmethyl, dimethylthiazolylmethyl, dimethylpyrazolylmethyl, methyloxadiazolylmethyl and methylpyridinylmethyl.

Suitable values of R^{b} include hydrogen and methylpyridinylmethyl.

In one embodiment, R^{b} represents hydrogen. In another embodiment, R^{b} is other than hydrogen.

Selected values of R^{c} include hydrogen; or C₁₋₆ alkyl, C₃₋₇ cycloalkyl or C₃₋₇ heterocycloalkyl, any of which groups may be optionally substituted by one or more substituents.

In a particular aspect, R^{c} represents hydrogen, C₁₋₆ alkyl or C₃₋₇ cycloalkyl.

Representative values of R^{c} include hydrogen; or methyl, cyclobutyl, cyclopentyl, cyclohexyl, tetrahydropyranyl and piperidinyl, any of which groups may be optionally substituted by one or more substituents.

Selected examples of suitable substituents on R^{c} include C₂₋₆ alkylcarbonyl and C₂₋₆ alkoxycarbonyl.

Selected examples of specific substituents on R^{c} include acetyl and tert-butoxycarbonyl.

Specific values of R^{c} include hydrogen, methyl, cyclobutyl, cyclopentyl, cyclohexyl, tetrahydropyranyl, acetylpiperidinyl and tert-butoxycarbonylpiperidinyl.

Suitably, R^{c} represents hydrogen or C₁₋₆ alkyl. In one embodiment, R^{c} is hydrogen. In another embodiment, R^{c} represents C₁₋₆ alkyl, especially methyl or ethyl, particularly methyl. In a further embodiment, R^{c} represents C₃₋₇ cycloalkyl, e.g. cyclopropyl, cyclobutyl, cyclopentyl or cyclohexyl.

Alternatively, the moiety —NR^{b}R^{c} may suitably represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents.

Selected examples of suitable substituents on the heterocyclic moiety —NR^{b}R^{c} include C₁₋₆ alkyl, C₁₋₆ alkylsulfonyl, hydroxy, hydroxy(C₁₋₆)alkyl, amino(C₁₋₆)alkyl, cyano, oxo, C₂₋₆ alkylcarbonyl, carboxy, C₂₋₆ alkoxycarbonyl, amino, C₂₋₆ alkylcarbonyl-amino, C₂₋₆ alkylcarbonylamino(C₁₋₆)alkyl, C₂₋₆ alkoxycarbonylamino, C₁₋₆ alkyl-sulfonylamino and aminocarbonyl.

Selected examples of specific substituents on the heterocyclic moiety —NR^{b}R^{c} include methyl, methylsulfonyl, hydroxy, hydroxymethyl, aminomethyl, cyano, oxo, acetyl, carboxy, ethoxycarbonyl, amino, acetylamino, acetylaminomethyl, tert-butoxy-carbonylamino, methylsulfonylamino and aminocarbonyl.

Specific values of the moiety —NR^{b}R^{c} include azetidin-1-yl, hydroxyazetidin-1-yl, hydroxymethylazetidin-1-yl, (hydroxy)(hydroxymethyl)azetidin-1-yl, aminomethyl-azetidin-1-yl, cyanoazetidin-1-yl, carboxyazetidin-1-yl, aminoazetidin-1-yl, aminocarbonylazetidin-1-yl, pyrrolidin-1-yl, aminomethylpyrrolidin-1-yl, oxopyrrolidin-1-yl, acetylaminomethylpyrrolidin-1-yl, tert-butoxycarbonylaminopyrrolidin-1-yl, oxo-oxazolidin-3-yl, hydroxyisoxazolidin-2-yl, thiazolidin-3-yl, oxothiazolidin-3-yl, dioxo-isothiazolidin-2-yl, piperidin-1-yl, hydroxypiperidin-1-yl, hydroxymethylpiperidin-1-yl, aminopiperidin-1-yl, acetylaminopiperidin-1-yl, tert-butoxycarbonylaminopiperidin-1-yl, methylsulfonylaminopiperidin-1-yl, morpholin-4-yl, piperazin-1-yl, methylpiperazin-1-yl, methylsulfonylpiperazin-1-yl, oxopiperazin-1-yl, acetylpiperazin-1-yl, ethoxycarbonyl-piperazin-1-yl and oxohomopiperazin-1-yl.

Suitably, R^{d} represents hydrogen; or C₁₋₆ alkyl, aryl or heteroaryl, any of which groups may be optionally substituted by one or more substituents.

Selected examples of suitable values for R^{d} include hydrogen, methyl, ethyl, isopropyl, 2-methylpropyl, tert-butyl, cyclopropyl, cyclobutyl, phenyl, thiazolidinyl, thienyl, imidazolyl and thiazolyl, any of which groups may be optionally substituted by one or more substituents.

Selected examples of suitable substituents on R^{d} include halogen, C₁₋₆ alkyl, C₁₋₆ alkoxy, oxo, C₂₋₆ alkylcarbonyloxy and di(C₁₋₆)alkylamino.

Selected examples of particular substituents on R^{d} include fluoro, methyl, methoxy, oxo, acetoxy and dimethylamino.

In one embodiment, R^{d} represents hydrogen. In another embodiment, R^{d} represents optionally substituted C₁₋₆ alkyl. In one aspect of that embodiment, R^{d} ideally represents unsubstituted C₁₋₆ alkyl, e.g. methyl, ethyl, isopropyl, 2-methylpropyl or tert-butyl, especially methyl or ethyl, particularly methyl. In another aspect of that embodiment, R^{d} ideally represents substituted C₁₋₆ alkyl, e.g. substituted methyl or substituted ethyl, including acetoxymethyl, dimethylaminomethyl and trifluoroethyl. In another embodiment, R^{d} represents optionally substituted aryl. In one aspect of that embodiment, R^{d} represents unsubstituted aryl, especially phenyl. In another aspect of that embodiment, R^{d} represents monosubstituted aryl, especially methylphenyl. In a further aspect of that embodiment, R^{d} represents disubstituted aryl, e.g. dimethoxyphenyl. In a further embodiment, R^{d} represents optionally substituted heteroaryl, e.g. thienyl, chlorothienyl, methylthienyl, methylimidazolyl or thiazolyl. In another embodiment, R^{d} represents optionally substituted C₃₋₇ cycloalkyl, e.g. cyclopropyl or cyclobutyl. In a further embodiment, R^{d} represents optionally substituted C₃₋₇ heterocycloalkyl, e.g. thiazolidinyl or oxothiazolidinyl.

Selected examples of specific values for R^{d} include hydrogen, methyl, ethyl, acetoxymethyl, dimethylaminomethyl, ethyl, trifluoroethyl, isopropyl, 2-methylpropyl, tert-butyl, cyclopropyl, cyclobutyl, phenyl, dimethoxyphenyl, thiazolidinyl, oxothiazolidinyl, thienyl, chlorothienyl, methylthienyl, methylimidazolyl and thiazolyl.

Appositely, R^{d} represents hydrogen or C₁₋₆ alkyl.

Individual values of R^{d} include hydrogen, methyl and ethyl.

A particular value of R^{d} is ethyl.

Suitably, R^{c} represents C₁₋₆ alkyl or aryl, either of which groups may be optionally substituted by one or more substituents.

Selected examples of suitable substituents on R^{e} include C₁₋₆ alkyl, especially methyl.

In one embodiment, R^{e} represents optionally substituted C₁₋₆ alkyl, ideally unsubstituted C₁₋₆ alkyl, e.g. methyl or propyl, especially methyl. In another embodiment, R^{e} represents optionally substituted aryl. In one aspect of that embodiment, R^{e} represents unsubstituted aryl, especially phenyl. In another aspect of that embodiment, R^{e} represents monosubstituted aryl, especially methylphenyl. In a further embodiment, R^{e} represents optionally substituted heteroaryl.

Selected values of R^{e} include methyl, propyl and methylphenyl.

One sub-class of compounds according to the invention is represented by the compounds of formula (IIA), and pharmaceutically acceptable salts and solvates thereof:

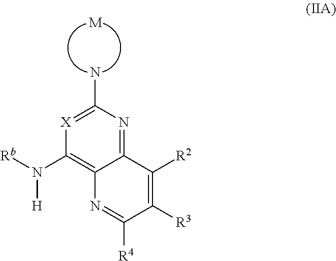

wherein

X, M, R², R³, R⁴ and R^{b} are as defined above.

Specific novel compounds in accordance with the present invention include each of the compounds whose preparation is described in the accompanying Examples, and pharmaceutically acceptable salts and solvates thereof.

The compounds in accordance with the present invention are beneficial in the treatment and/or prevention of various human ailments. These include inflammatory, autoimmune and oncological disorders; viral diseases and malaria; and organ and cell transplant rejection.

Inflammatory and autoimmune disorders include systemic autoimmune disorders, autoimmune endocrine disorders and organ-specific autoimmune disorders. Systemic autoimmune disorders include systemic lupus erythematosus (SLE), psoriasis, vasculitis, polymyositis, scleroderma, multiple sclerosis, ankylosing spondylitis, rheumatoid arthritis and Sjögren's syndrome. Autoimmune endocrine disorders include thyroiditis. Organ-specific autoimmune disorders include Addison's disease, haemolytic or pernicious anaemia, glomerulonephritis (including Goodpasture's syndrome), Graves' disease, idiopathic thrombocytopenic purpura, insulin-dependent diabetes mellitus, juvenile diabetes, uveitis, inflammatory bowel disease (including Crohn's disease and ulcerative colitis), pemphigus, atopic dermatitis, autoimmune hepatitis, primary biliary cirrhosis, autoimmune pneumonitis, autoimmune carditis, myasthenia gravis and spontaneous infertility.

Oncological disorders, which may be acute or chronic, include proliferative disorders, especially cancer, in animals, including mammals, especially humans. Particular categories of cancer include haematological malignancy (including leukaemia and lymphoma) and non-haematological malignancy (including solid tumour cancer, sarcoma, meningioma, glioblastoma multiforme, neuroblastoma, melanoma, gastric carcinoma and renal cell carcinoma). Chronic leukaemia may be myeloid or lymphoid. Varieties of leukaemia include lymphoblastic T cell leukaemia, chronic myelogenous leukaemia (CML), chronic lymphocytic/lymphoid leukaemia (CLL), hairy-cell leukaemia, acute lymphoblastic leukaemia (ALL), acute myelogenous leukaemia (AML), myelodysplastic syndrome, chronic neutrophilic leukaemia, acute lymphoblastic T cell leukaemia, plasmacytoma, immunoblastic large cell leukaemia, mantle cell leukaemia, multiple myeloma, acute megakaryoblastic leukaemia, acute megakaryocytic leukaemia, promyelocytic leukaemia and erythroleukaemia. Varieties of lymphoma include malignant lymphoma, Hodgkin's lymphoma, non-Hodgkin's lymphoma, lymphoblastic T cell lymphoma, Burkitt's lymphoma, follicular lymphoma, MALT1 lymphoma and marginal zone lymphoma. Varieties of non-haematological malignancy include cancer of the prostate, lung, breast, rectum, colon, lymph node, bladder, kidney, pancreas, liver, ovary, uterus, cervix, brain, skin, bone, stomach and muscle.

Viral diseases include infections caused by various families of virus, including the Retroviridae, Flaviviridae, Picornaviridae. Various genera within the Retroviridae family include Alpharetrovirus, Betaretrovirus, Gammaretrovirus, Deltaretrovirus, Epsilonretrovirus, Lentivirus and Spumavirus. Members of the Lentivirus genus include human immunodeficiency virus 1 (HIV-1) and human immunodeficiency virus 2 (HIV-2). Various genera within the Flaviviridae family include Flavivirus, Pestivirus, Hepacivirus and Hepatitis G Virus. Members of the Flavivirus genus include Dengue fever virus, yellow fever virus, West Nile encephalitis virus and Japanese encephalitis virus. Members of the Pestivirus genus include bovine viral diarrhoea virus (BVDV), classical swine fever virus and border disease virus 2 (BDV-2). Members of the Hepacivirus genus include hepatitis C virus (HCV). Members of the Hepatitis G Virus genus include hepatitis G virus. Various genera within the Picornaviridae family include Aphthovirus, Avihepatovirus, Cardiovirus, Enterovirus, Erbovirus, Hepatovirus, Kobuvirus, Parechovirus, Sapelovirus, Senecavirus, Teschovirus and Tremovirus. Members of the Enterovirus genus include poliovirus, coxsackie A virus, coxsackie B virus and rhinovirus.

Organ transplant rejection includes the rejection of transplanted or grafted organs or cells (both allografts and xenografts), including graft-versus-host reaction disease. The term “organ” as used herein means all organs or parts of organs in mammals, particularly humans, including kidney, lung, bone marrow, hair, cornea, eye (vitreous), heart, heart valve, liver, pancreas, blood vessel, skin, muscle, bone, intestine and stomach. The term “rejection” as used herein means all reactions of the recipient body or the transplanted organ which ultimately lead to cell or tissue death in the transplanted organ, or adversely affect the functional ability and viability of the transplanted organ or the recipient. In particular, this means acute and chronic rejection reactions.

Cell transplant rejection includes the rejection of cell transplants and xeno-transplantation. The major hurdle for xenotransplantation is that even before the T lymphocytes (responsible for the rejection of allografts) are activated, the innate immune system (especially T-independent B lymphocytes and macrophages) is activated. This provokes two types of severe and early acute rejection, referred to as hyperacute rejection and vascular rejection respectively. Conventional immunosuppressant drugs, including cyclosporine A, are ineffective in xenotransplantation. The compounds in accordance with the present invention are not liable to this drawback. The ability of the compounds of this invention to suppress T-independent xeno-antibody production as well as macrophage activation may be demonstrated by their ability to prevent xenograft rejection in athymic, T-deficient mice receiving xenogenic hamster-heart grafts.

The present invention also provides a pharmaceutical composition which comprises a compound in accordance with the invention as described above, or a pharmaceutically acceptable salt or solvate thereof, in association with one or more pharmaceutically acceptable carriers.

Pharmaceutical compositions according to the invention may take a form suitable for oral, buccal, parenteral, nasal, topical, ophthalmic or rectal administration, or a form suitable for administration by inhalation or insufflation.

For oral administration, the pharmaceutical compositions may take the form of, for example, tablets, lozenges or capsules prepared by conventional means with pharmaceutically acceptable excipients such as binding agents (e.g. pregelatinised maize starch, polyvinylpyrrolidone or hydroxypropyl methyl cellulose); fillers (e.g. lactose, microcrystalline cellulose or calcium hydrogenphosphate); lubricants (e.g. magnesium stearate, talc or silica); disintegrants (e.g. potato starch or sodium glycollate); or wetting agents (e.g. sodium lauryl sulfate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents, emulsifying agents, non-aqueous vehicles or preservatives. The preparations may also contain buffer salts, flavouring agents, colouring agents or sweetening agents, as appropriate.

Preparations for oral administration may be suitably formulated to give controlled release of the active compound.

For buccal administration, the compositions may take the form of tablets or lozenges formulated in conventional manner.

The compounds of formula (I) may be formulated for parenteral administration by injection, e.g. by bolus injection or infusion. Formulations for injection may be presented in unit dosage form, e.g. in glass ampoules or multi-dose containers, e.g. glass vials. The compositions for injection may take such forms as suspensions, solutions or emulsions in oily or aqueous vehicles, and may contain formulatory agents such as suspending, stabilising, preserving and/or dispersing agents. Alternatively, the active ingredient may be in powder form for constitution with a suitable vehicle, e.g. sterile pyrogen-free water, before use.

In addition to the formulations described above, the compounds of formula (I) may also be formulated as a depot preparation. Such long-acting formulations may be administered by implantation or by intramuscular injection.

For nasal administration or administration by inhalation, the compounds according to the present invention may be conveniently delivered in the form of an aerosol spray presentation for pressurised packs or a nebuliser, with the use of a suitable propellant, e.g. dichlorodifluoromethane, fluorotrichloromethane, dichlorotetrafluoroethane, carbon dioxide or other suitable gas or mixture of gases.

The compositions may, if desired, be presented in a pack or dispenser device which may contain one or more unit dosage forms containing the active ingredient. The pack or dispensing device may be accompanied by instructions for administration.

For topical administration the compounds of use in the present invention may be conveniently formulated in a suitable ointment containing the active component suspended or dissolved in one or more pharmaceutically acceptable carriers. Particular carriers include, for example, mineral oil, liquid petroleum, propylene glycol, polyoxyethylene, polyoxypropylene, emulsifying wax and water. Alternatively, the compounds of use in the present invention may be formulated in a suitable lotion containing the active component suspended or dissolved in one or more pharmaceutically acceptable carriers. Particular carriers include, for example, mineral oil, sorbitan monostearate, polysorbate 60, cetyl esters wax, cetearyl alcohol, benzyl alcohol, 2-octyldodecanol and water.

For ophthalmic administration the compounds of use in the present invention may be conveniently formulated as micronized suspensions in isotonic, pH-adjusted sterile saline, either with or without a preservative such as a bactericidal or fungicidal agent, for example phenylmercuric nitrate, benzylalkonium chloride or chlorhexidine acetate. Alternatively, for ophthalmic administration compounds may be formulated in an ointment such as petrolatum.

For rectal administration the compounds of use in the present invention may be conveniently formulated as suppositories. These can be prepared by mixing the active component with a suitable non-irritating excipient which is solid at room temperature but liquid at rectal temperature and so will melt in the rectum to release the active component. Such materials include, for example, cocoa butter, beeswax and polyethylene glycols.

The quantity of a compound of use in the invention required for the prophylaxis or treatment of a particular condition will vary depending on the compound chosen and the condition of the patient to be treated. In general, however, daily dosages may range from around 10 ng/kg to 1000 mg/kg, typically from 100 ng/kg to 100 mg/kg, e.g. around 0.01 mg/kg to 40 mg/kg body weight, for oral or buccal administration, from around 10 ng/kg to 50 mg/kg body weight for parenteral administration, and from around 0.05 mg to around 1000 mg, e.g. from around 0.5 mg to around 1000 mg, for nasal administration or administration by inhalation or insufflation.

The compounds of formula (I) above may be prepared by a process which comprises reacting a compound of formula (III) with a compound of formula (IV):

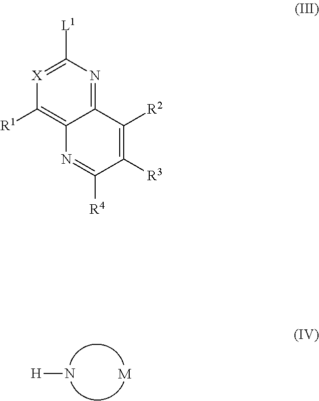

wherein X, M, R¹, R², R³ and R⁴ are as defined above, and L¹ represents a suitable leaving group.

The leaving group L¹ is typically a halogen atom, e.g. chloro. Alternatively, the leaving group L¹ may be C₁₋₆ alkylsulfanyl, e.g. methylsulfanyl, or C₁₋₆ alkylsulfonyl, e.g. methylsulfonyl.

The reaction is conveniently effected at an elevated temperature in a suitable solvent, e.g. an organic nitrile such as acetonitrile, a lower alkanol such as ethanol, isopropanol or n-butanol, an ethereal solvent such as tetrahydrofuran or 1,4-dioxane, or an organic amide such as N,N-dimethylacetamide or 1-methyl-2-pyrrolidinone. The reaction may be performed in the presence of a suitable base, e.g. an organic base such as N,N-diisopropylethylamine or 1,8-diazabicyclo[5.4.0]undec-7-ene.

Alternatively, the reaction may be performed in the presence of a transition metal catalyst. The transition metal catalyst is suitably a palladium-containing catalyst such as bis(tri-tert-butylphosphine)palladium(0). The reaction is conveniently effected at an elevated temperature in a suitable solvent, e.g. an ethereal solvent such as 1,4-dioxane, typically in the presence of cesium carbonate.

The compounds of formula (I) above, wherein R² represents optionally substituted aryl or optionally substituted heteroaryl, may be prepared by a process which comprises reacting a compound of formula R^{2a}—B¹ with a compound of formula (V):

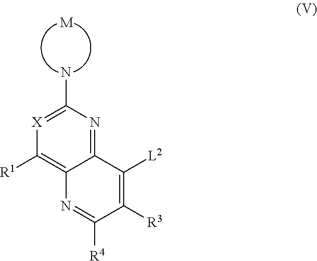

wherein X, M, R¹, R³ and R⁴ are as defined above, R^{2a} represents optionally substituted aryl or optionally substituted heteroaryl, L² represents a suitable leaving group, and B¹ represents a boronic acid moiety —B(OH)₂ or a cyclic ester thereof formed with an organic diol, e.g. pinacol, 1,3-propanediol or neopentyl glycol; in the presence of a transition metal catalyst.

The leaving group L² is typically a halogen atom, e.g. bromo or iodo.

The transition metal catalyst of use in the reaction between the compound of formula R^{2a}—B¹ and compound (V) is suitably a palladium-containing catalyst such as tetrakis(triphenylphosphine)palladium(0) or dichloro[1,1′-bis(diphenylphosphino)-ferrocene]palladium(II).

The reaction is conveniently carried out at an elevated temperature in a suitable solvent, e.g. an ethereal solvent such as 1,4-dioxane or 1,2-dimethoxyethane, typically in the presence of potassium phosphate, potassium carbonate or sodium carbonate.

The intermediates of formula (V) may be prepared by reacting a compound of formula (IV) as defined above with a compound of formula (VI):

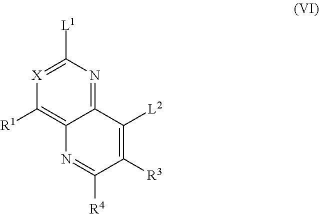

wherein X, R¹, R³, R⁴, L¹ and L² are as defined above; under conditions analogous to those described above for the reaction between compounds (III) and (IV).

An intermediate of formula (III) or (VI) wherein L¹ represents C₁₋₆ alkylsulfanyl, e.g. methylsulfanyl, may be converted into the corresponding compound wherein L¹ represents C₁₋₆ alkylsulfonyl, e.g. methylsulfonyl, by treatment with a suitable oxidising agent, e.g. 3-chloroperoxybenzoic acid.

The intermediates of formula (VI) wherein R¹ represents —NR^{b}R^{c} may be prepared by reacting a compound of formula H—NR^{b}R^{c} with a compound of formula (VII):

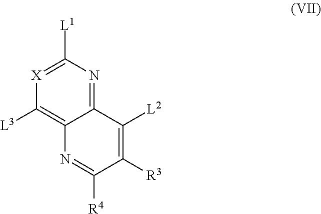

wherein X, R³, R⁴, R^{b}, R^{c}, L¹ and L² are as defined above, and L³ represents a suitable leaving group.

The leaving group L³ is typically a halogen atom, e.g. chloro.

The reaction is conveniently effected at an elevated temperature in a suitable solvent, e.g. a lower alkanol such as isopropanol or n-butanol, or an organic amide such as 1-methyl-2-pyrrolidinone. The reaction may be performed in the presence of a suitable base, e.g. an organic base such as N,N-diisopropylethylamine. By analogy, where R^{b} and R^{c} are both H, the reaction may conveniently be performed by treating compound (VII) with aqueous ammonia, or aqueous ammonium hydroxide solution, in a suitable solvent, e.g. an ethereal solvent such as 1,4-dioxane.

The intermediates of formula (VI) and (VII) wherein L² represents a halogen atom, e.g. bromo or iodo, may be prepared by reacting a compound of formula (VIII) or (IX) respectively:

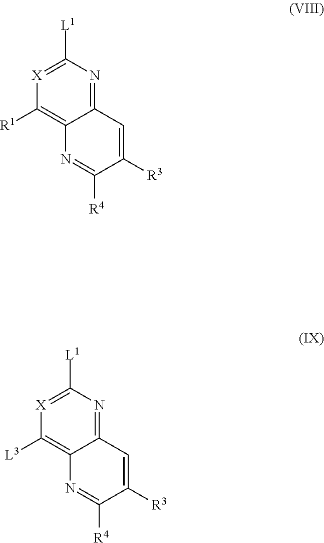

wherein X, R¹, R³, R⁴, L¹ and L³ are as defined above; with a halogenating agent, e.g. elemental bromine or N-iodosuccinimide.

The intermediates of formula (III) wherein R¹ represents —NR^{b}R^{c} may be prepared by reacting a compound of formula H—NR^{b}R^{c} with a compound of formula (X):

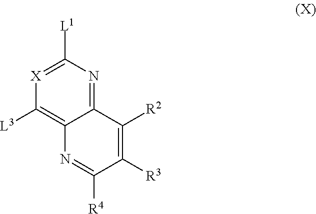

wherein X, R², R³, R⁴, R^{b}, R^{c}, L¹ and L³ are as defined above; under conditions analogous to those described above for the reaction between a compound of formula H—NR^{b}R^{c} and compound (VII).

As will be appreciated, the intermediates of formula (V) above wherein L² represents halogen correspond to compounds in accordance with the present invention wherein R² represents halogen.

Where they are not commercially available, the starting materials of formula (IV), (VIII), (IX) and (X) may be prepared by methods analogous to those described in the accompanying Examples, or by standard methods well known from the art.

It will be understood that any compound of formula (I) initially obtained from any of the above processes may, where appropriate, subsequently be elaborated into a further compound of formula (I) by techniques known from the art. By way of example, a compound comprising a N—BOC moiety may be converted into the corresponding compound comprising a N—H moiety by treatment with an acid, e.g. a mineral acid such as hydrochloric acid, or an organic acid such as trifluoroacetic acid.

A compound wherein R¹ represents halogen, e.g. chloro, may be converted into the corresponding compound wherein R¹ represents amino (—NH₂) in a two-step procedure which comprises: (i) treatment with benzylamine; and (ii) removal of the benzyl moiety from the material thereby obtained by catalytic hydrogenation. Alternatively, a compound wherein R¹ represents halogen, e.g. chloro, may be converted into the corresponding compound wherein R¹ represents amino (—NH₂) in a two-step procedure which comprises: (i) treatment with 4-methoxybenzylamine; and (ii) removal of the 4-methoxybenzyl moiety from the material thereby obtained by treatment with acid, e.g. an organic acid such as trifluoroacetic acid.

A compound wherein R¹ represents —SR^{a} may be converted into the corresponding compound wherein R¹ represents —SO₂R^{a} by treatment with an oxidising agent, typically 3-chloroperoxybenzoic acid (MCPBA).

A compound wherein R¹ represents —SO₂R^{a}, e.g. methylsulfonyl, may be converted into the corresponding compound wherein R¹ represents —OR^{a} by treatment with a sodium salt of formula NaOR^{a}. Similarly, a compound wherein R¹ represents —SO₂R^{a}, e.g. methylsulfonyl, may be converted into the corresponding compound wherein R¹ represents cyano by treatment with a cyanide salt, e.g. an alkali metal cyanide salt such as sodium cyanide. Likewise, a compound wherein R¹ represents —SO₂R^{a}, e.g. methylsulfonyl, may be converted into the corresponding compound wherein R¹ represents —NR^{b}R^{c} by treatment with an amine of formula H—NR^{b}R^{c}. By analogy, a compound wherein R¹ represents —SO₂R^{a}, e.g. methylsulfonyl, may be converted into the corresponding compound wherein R¹ represents —NH₂ by treatment with ammonium hydroxide.

A compound wherein R¹ represents —NR^{c}OR^{d} may be converted into the corresponding compound wherein R¹ represents —NHR^{c} by treatment with a base, typically an alkali metal carbonate such as potassium carbonate.

A compound wherein R² represents —CO₂R^{d}, in which R^{d} is other than hydrogen, may be converted into the corresponding compound wherein R² represents carboxy (—CO₂H) by treatment with a base, typically an alkali metal hydroxide such as sodium hydroxide.

A compound wherein R² represents carboxy (—CO₂H) may be converted into the corresponding compound wherein R² represents —CONR^{b}R^{c} or —CON(OR^{a})R^{b} by treatment with the appropriate reagent of formula H—NR^{b}R^{c} or H—N(OR^{a})R^{b} respectively. The reaction may typically be performed in the presence of a coupling agent such as 1-(3-dimethylaminopropyl)-3-ethylcarbodiimide hydrochloride (EDC) and an additive such as 1-hydroxybenzotriazole hydrate (HOBT), optionally in the presence of a base, e.g. an organic base such as N,N-diisopropylethylamine. Alternatively, the reaction may be performed in the presence of a coupling agent such as O-(benzotriazol-1-yl)-N,N,N′,N′-tetramethyluronium tetrafluoroborate (TBTU) and a base, e.g. an organic base such as N,N-diisopropylethylamine.

A compound wherein R² represents carboxy (—CO₂H) may be converted into the corresponding compound wherein R² represents —CONH₂ by treatment with ammonium chloride, typically in the presence of a coupling agent such as EDC and an additive such as HOBT, suitably in the presence of a base, e.g. an organic base such as diisopropylamine or N,N-diisopropylethylamine. A compound wherein R² represents —CONH₂ may be converted into the corresponding compound wherein R² represents cyano (—CN) by treatment with phosphorus oxychloride. Alternatively, a compound wherein R² represents —CONH₂ may be converted into the corresponding compound wherein R² represents cyano in a two-step procedure which comprises: (i) treatment with cyanuric chloride; and (ii) treatment of the material thereby obtained with water.

A compound wherein R² represents carboxy (—CO₂H) may be converted into the corresponding compound wherein R² represents hydrogen by heating in the presence of a base, e.g. an organic amine such as triethylamine.

A compound wherein R² represents carboxy (—CO₂H) may be converted into the corresponding compound wherein R² represents hydroxymethyl (—CH₂OH) in a two-step procedure which comprises: (i) treatment with ethyl chloroformate and triethylamine; and (ii) treatment of the material thereby obtained with a reducing agent, typically an alkali metal borohydride such as sodium borohydride.

A compound wherein R² represents carboxy (—CO₂H) may be converted into the corresponding compound wherein R² represents hydroxy in a two-step procedure which comprises: (i) treatment with diphenyl phosphoryl azide; and (ii) treatment of the material thereby obtained with water.

A compound wherein R² represents carboxy (—CO₂H) may be converted into the corresponding compound wherein R² represents —NHCO₂R^{d}, wherein R^{d} is other than hydrogen, in a two-step procedure which comprises: (i) treatment with diphenyl phosphoryl azide; and (ii) treatment of the material thereby obtained with the appropriate reagent of formula R^{d}—OH.

A compound wherein R² represents carboxy (—CO₂H) may be converted into the corresponding compound wherein R² represents a 3-substituted 1,2,4-oxadiazol-5-yl moiety in a two-step procedure which comprises: (i) treatment with an appropriately-substituted N′-hydroxyamidine derivative, typically in the presence of a coupling agent such as O-(7-azabenzotriazol-1-yl)-N,N,N′,N′-tetramethyluronium hexafluorophosphate (HATU), suitably in the presence of a base, e.g. an organic base such as N,N-diisopropyl-ethylamine; and (ii) treatment of the material thereby obtained with a strong base, suitably a strong inorganic base, e.g. an alkali metal tert-butoxide such as potassium tert-butoxide.

A compound wherein R² represents 4,5-dihydrooxazol-2-yl may be prepared from the corresponding compound wherein R² represents —CONR^{b}R^{c}, in which R^{b} represents —CH₂CH₂OH and R^{c} represents hydrogen, by heating with a condensing agent such as N,N′-diisopropylcarbodiimide, typically in the presence of copper(II) trifluoromethane-sulfonate.

Where a mixture of products is obtained from any of the processes described above for the preparation of compounds according to the invention, the desired product can be separated therefrom at an appropriate stage by conventional methods such as preparative HPLC; or column chromatography utilising, for example, silica and/or alumina in conjunction with an appropriate solvent system.

Where the above-described processes for the preparation of the compounds according to the invention give rise to mixtures of stereoisomers, these isomers may be separated by conventional techniques. In particular, where it is desired to obtain a particular enantiomer of a compound of formula (I) this may be produced from a corresponding mixture of enantiomers using any suitable conventional procedure for resolving enantiomers. Thus, for example, diastereomeric derivatives, e.g. salts, may be produced by reaction of a mixture of enantiomers of formula (I), e.g. a racemate, and an appropriate chiral compound, e.g. a chiral base. The diastereomers may then be separated by any convenient means, for example by crystallisation, and the desired enantiomer recovered, e.g. by treatment with an acid in the instance where the diastereomer is a salt. In another resolution process a racemate of formula (I) may be separated using chiral HPLC. Moreover, if desired, a particular enantiomer may be obtained by using an appropriate chiral intermediate in one of the processes described above. Alternatively, a particular enantiomer may be obtained by performing an enantiomer-specific enzymatic biotransformation, e.g. an ester hydrolysis using an esterase, and then purifying only the enantiomerically pure hydrolysed acid from the unreacted ester antipode. Chromatography, recrystallisation and other conventional separation procedures may also be used with intermediates or final products where it is desired to obtain a particular geometric isomer of the invention.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} edition, 1999. The protecting groups may be removed at any convenient subsequent stage utilising methods known from the art.

The following Examples illustrate the preparation of compounds according to the invention.

The compounds in accordance with this invention potently inhibit the activity of human PI4KIIIβ.

### PI4KIIIβ Enzyme Inhibition Assay

### Procedure A

Compounds were assayed utilizing reagents from Invitrogen and Promega. Compounds were screened in 1% DMSO (final) as 3-fold serial dilutions from a starting concentration of 20 μM. The 2.5× PI4Kβ reagent, the 2.5×PI Lipid Kinase Substrate/ATP mixture and the 5× compounds were prepared in 20 mM Tris pH 7.5, 0.5 mM EGTA, 2 mM DTT, 5 mM MgCl₂, 0.4% Triton. The final 25 μL Kinase Reaction consisted of: 4 nM PI4Kβ, 100 μM PI Lipid Kinase Substrate (both Invitrogen), and compound. The final ATP concentration in the assay was 10 μM. The detection reagents consisted of ADP-Glo™ Reagent and ADP-Glo™ Detect Reagent (Promega).

Briefly, compound was added to PI4Kβ followed by addition of ATP/PI Lipid Kinase Substrate mixture. The reaction mixture was incubated for 60 minutes at room temperature. The ADP-Glo™ Reagent was added and the plate was incubated for 40 minutes at room temperature, followed by addition of ADP-Glo™ Detect Reagent. The plate was incubated for a further 120 minutes and read on a Luminescence plate reader. The data was fitted with XLfit from IDBS using model number 205.

### Procedure B

Compounds were assayed using a PI4Kbeta Adapta assay. Compounds were screened in 1% DMSO (final) as 3-fold serial dilutions from a starting concentration of 10 μM. The 2× PI4KB (PI4K beta)/PI Lipid Kinase Substrate mixture was prepared in 50 mM HEPES pH 7.5, 0.1% CHAPS, 1 mM EGTA, 4 mM MgCl₂. The final 10 μL Kinase Reaction consisted of 7.5-60 ng PI4Kβ, and 100 μM PI Lipid Kinase Substrate in 32.5 mM HEPES pH 7.5, 0.05% CHAPS, 0.5 mM EGTA, 2 mM MgCl₂. The final ATP concentration in the assay was 10 μM. The detection mix consisted of EDTA (30 mM), Eu-anti-ADP antibody (6 nM) and ADP tracer. The detection mix contained the EC60 concentration of tracer for 5-150 μM ATP.

Briefly, ATP was added to compound, followed by addition of a PI4Kβ/PI Lipid Kinase Substrate mixture. The plate was shaken for 30 seconds to mix, then briefly centrifuged. The reaction mixture was incubated for 60 minutes at room temperature. The detection mix was added, then the plate was shaken and centrifuged. The plate was incubated for 60 minutes at room temperature and read on a fluorescence plate reader. The data was fitted with XLfit from IDBS using model number 205.

When tested in the above assay (Procedure A or Procedure B), the compounds of the accompanying Examples were all found to possess IC₅₀ values for inhibition of the activity of human PI4KIIIβ of 50 μM or better.

Certain compounds in accordance with this invention are potent inhibitors when measured in the MLR test described below.

### The Mixed Lymphocyte Reaction (MLR) Test

Human peripheral blood mononuclear cells (PBMCs) were isolated from buffy coats, obtained from healthy blood donors by Ficoll (Lymphoprep, Axis-Shield PoC AS, Oslo, Norway) density-gradient centrifugation. The cells at the Ficoll-plasma interface were washed three times and used as “Responder” cells. RPMI 1788 (ATCC, N° CCL-156) cells were treated with mitomycin C (Kyowa, Nycomed, Brussels, Belgium) and used as “Stimulator” cells. Responder cells (0.12×106), Stimulator cells (0.045×106) and compounds (in different concentrations) were cocultured for 6 days in RPMI 1640 medium (BioWhittaker, Lonza, Belgium) supplemented with 10% fetal calf serum, 100 U/ml Geneticin (Gibco, LifeTechnologies, UK). Cells were cultured in triplicate in flat-bottomed 96-well microtiter tissue culture plates (TTP, Switzerland). After 5 days, cells were pulsed with 1 μCi of methyl-³H thymidine (MP Biomedicals, USA), harvested 18 h later on glass filter paper and counted. Proliferation values were expressed as counts per minute (cpm), and converted to % inhibition with respect to a blank MLR test (identical but without added compound). The IC₅₀ was determined from a graph with at least four points, each derived from the mean of 2 experiments. The IC₅₀ value represents the lowest concentration of test compound (expressed in μM) that resulted in a 50% inhibition of the MLR.

Certain compounds of the accompanying Examples were found to generate IC₅₀ values in the MLR test of 10 μM or better.

### EXAMPLES

### Abbreviations

THF: tetrahydrofuran MeOH: methanol

DMF: N,N-dimethylformamide DMSO: dimethyl sulfoxide

DCM: dichloromethane DIPEA: N,N-diisopropylethylamine

EtOAc: ethyl acetate n-BuOH: butan-1-ol

Et₂O: diethyl ether TFA: trifluoroacetic acid

h: hour r.t.: room temperature

MS: Mass Spectrometry M: mass

LCMS: Liquid Chromatography Mass Spectrometry

HPLC: High Performance Liquid Chromatography

ES+: Electrospray Positive Ionisation RT: retention time

### Analytical and Purification Methods

### Method 1

High pH (approximately pH 9.5)
Column: Waters XBridge, C18, 2.1×20 mm, 2.5 μm
Solvent A: 10 mM ammonium formate in water+0.1% ammonia solution
Solvent B: acetonitrile+5% solvent A+0.1% ammonia solution

### Gradient Program:

| Time | A % | B % |
|---|---|---|
| 0.00 | 95.0 | 5.0 |
| 1.50 | 5.0 | 95.0 |
| 2.50 | 5.0 | 95.0 |
| 3.00 | 95.0 | 5.0 |

### Method 2

High pH (approximately pH 9.5)
Column: Waters XBridge, C18, 2.1×20 mm, 2.5 μm
Solvent A: 10 mM ammonium formate in water+0.1% ammonia solution
Solvent B: acetonitrile+5% solvent A+0.1% ammonia solution

### Gradient Program:

| Time | A % | B % |
|---|---|---|
| 0.00 | 95.0 | 5.0 |
| 4.00 | 5.0 | 95.0 |
| 5.00 | 5.0 | 95.0 |
| 5.10 | 95.0 | 5.0 |

### Method 3

Low pH (approximately pH 3)
Column: Waters XBridge, C18, 2.1×20 mm, 2.5 μm
Solvent A: water+0.1% formic acid
Solvent B: acetonitrile+5% solvent A+0.1% formic acid

### Gradient Program:

| Time | A % | B % |
|---|---|---|
| 0.00 | 95.0 | 5.0 |
| 4.00 | 5.0 | 95.0 |
| 5.00 | 5.0 | 95.0 |
| 5.10 | 95.0 | 5.0 |

### Method 4

High pH (approximately pH 9.5)
Column: Waters Acquity UPLC BEH, C18, 2.1×50 mm, 1.7 μm
Solvent A: 10 mM ammonium formate in water+0.1% ammonia solution
Solvent B: acetonitrile+5% solvent A+0.1% ammonia solution

### Gradient Program:

| Time | A % | B % |
|---|---|---|
| 0.00 | 98.0 | 2.0 |
| 4.00 | 5.0 | 95.0 |
| 5.00 | 5.0 | 95.0 |
| 5.10 | 98.0 | 2.0 |

### Intermediate 1

### tert-Butyl (3S)-4-(4-aminopyrido[3,2-c]pyrimidin-2-yl)-3-methylpiperazine-1-carboxylate

To a solution of 2-chloropyrido[3,2-d]pyrimidin-4-amine (J. Chem. Soc., 1956, 1045-54) (750 mg, 4.17 mmol) in n-BuOH (5 mL) was added tert-butyl (3S)-3-methyl-piperazine-1-carboxylate (990 mg, 4.99 mmol), followed by DIPEA (1.8 mL, 12.4 mmol). The reaction mixture was heated at 130° C. for 16 h, then concentrated in vacuo and diluted with EtOAc (50 mL). The organic layer was washed with H₂O (15 mL) and brine, then dried over anhydrous sodium sulfate and concentrated in vacuo. The residue was purified by column chromatography (Normal Phase; 100-200 mesh silica; 30% EtOAc in hexanes) to afford the title compound (550 mg, 38%). δ_{H} (DMSO-d₆, 400 MHz) 8.38-8.40 (m, 1H), 7.60-7.65 (m, 2H), 7.52-7.54 (br, 2H), 4.85-4.88 (m, 1H), 4.52-4.60 (m, 1H), 3.85-4.05 (m, 1H), 3.78-3.82 (m, 1H), 3.00-3.10 (m, 2H), 2.80-2.90 (m, 1H), 1.40 (s, 9H), 1.23 (d, J6.6 Hz, 3H).

### Intermediate 2

### Phenyl N-[6-(dimethylamino)-2-methylpyridin-3-yl]carbamate

To a cooled (ice bath) solution of N²,N²,6-trimethylpyridine-2,5-diamine (1 mmol) in THF (50 mL) was added pyridine (1.1 equivalents), followed by phenyl chloroformate (1 equivalent) dropwise. The reaction mixture was allowed to warm to r.t. When LCMS confirmed complete conversion of the amine to the desired carbamate, the reaction mixture was quenched with water and and extracted into DCM, then phase separated and concentrated in vacuo. The residue was used without further purification. LCMS (ES+) [M+H]⁺ 272, RT 1.79 minutes (method 2).

### Intermediate 3

### 5-{[(4,6-Dichloropyridin-3-yl)amino]methylene}-2,2-dimethyl-1,3-dioxane-4,6-dione

A mixture of 5-amino-2,4-dichloropyridine (818 mg, 5.02 mmol) and 2,2-dimethyl-1,3-dioxane-4,6-dione (889 mg, 6.05 mmol) was heated to 100° C. After 2 minutes, trimethyl orthoformate (2.32 mL, 30.1 mmol) was added to the melt. A white suspension formed over 25 minutes and reflux was observed. The mixture was cooled to ambient temperature and the suspension was diluted with diethyl ether (10 mL). The reaction was filtered to afford the title compound (90% purity, 1.40 g, 79%) as an off-white powder. δ_{H} (DMSO-d₆, 300 MHz) 11.40 (d, J10.9 Hz, 1H), 8.92 (s, 1H), 8.86-8.73 (m, 1H), 7.99 (s, 1H), 1.70 (s, 6H). LCMS (ES-) [M−H]⁻ 315, RT 1.41 minutes (method 2).

### Intermediate 4

### 6,8-Dichloro-1,5-naphthyridin-4-ol

To hot (250° C.) diphenyl ether (15 mL) was added Intermediate 3 (1.17 g, 3.34 mmol) in 4 portions. After 5 minutes at 250° C. the dark brown solution was cooled to ambient temperature, then poured onto isohexane (60 mL). The precipitate was recovered on a sinter, dissolved in MeOH and dry-loaded onto silica. The crude material was purified using flash column chromatography on silica (gradient elution with 0-100% MeOH/DCM) to afford the title compound (419 mg, 53%) as a brown powder. δ_{H} (DMSO-d₆, 300 MHz) 11.80 (s, 1H), 8.54-7.66 (m, 2H), 6.41 (br s, 1H). LCMS (ES+) [M+H]⁺ 215, RT 0.39 minutes (method 2).

### Intermediate 5

### 8-Bromo-2,4-dichloro-1,5-naphthyridine

Intermediate 4 (200 mg, 0.84 mmol) was suspended in DMF (1.5 mL) and the mixture was cooled to 0° C. Phosphorus tribromide (0.11 mL, 1.2 mmol) was added dropwise and after 2 minutes the mixture was warmed to ambient temperature. The suspension turned from dark brown to pale brown. DMF (3 mL) was added to the suspension. After 25 minutes the mixture was poured onto a mixture of crushed ice and water (˜15 mL), then vigorously stirred for 2 minutes. The pH was adjusted to pH 7-8 using saturated sodium bicarbonate solution. The suspension was extracted with EtOAc (2×15 mL). The combined organic layers were washed with 50% saturated aqueous sodium chloride solution (2×20 mL) and saturated aqueous sodium chloride solution (20 mL), then dried over anhydrous sodium sulfate. Concentration in vacuo afforded the title compound (232 mg, quantitative) as a light brown solid. δ_{H} (DMSO-d₆, 300 MHz,) 8.90 (d, J 4.7 Hz, 1H), 8.36 (d, J 4.7 Hz, 1H), 8.35 (s, 1H). LCMS (ES+, ionisation not observed), RT 1.77 minutes (method 2).

### Intermediate 6

### 2,4-Dichloro-8-(3,4-dimethoxyphenyl)-1,5-naphthyridine

To a mixture of 3,4-dimethoxyphenylboronic acid (228 mg, 1.25 mmol), Intermediate 5 (317 mg, 1.14 mmol) and potassium phosphate tribasic (242 mg, 1.14 mmol) were added 1,4-dioxane (4.8 mL) and water (1.2 mL). The suspension was purged with nitrogen for 20 minutes before tetrakis(triphenylphosphine)palladium(0) (65.9 mg, 0.057 mmol) was added. The mixture was heated at 80° C. for 1.5 h, then at 150° C. for 15 minutes. The mixture was cooled to ambient temperature, diluted with EtOAc (40 mL) and washed with water (2×15 mL). Silica was added and the slurry was concentrated in vacuo. Purification using flash column chromatography on silica (gradient elution with 0-45% EtOAc/isohexane) afforded the title compound (80% purity, 304 mg, 64%) as a yellow solid. δ_{H} (DMSO-d₆, 300 MHz,) 9.08 (d, J4.5 Hz, 1H), 8.23 (s, 1H), 7.96 (d, J 4.6 Hz, 1H), 7.45 (d, J 2.1 Hz, 1H), 7.37 (dd, J 8.3, 2.1 Hz, 1H), 7.13 (d, J8.4 Hz, 1H), 3.85 (s, 3H), 3.83 (s, 3H). LCMS (ES+) [M+H]⁺ 335, RT 2.23 minutes (method 2).

### Intermediate 7

### 2-Chloro-8-(3,4-dimethoxyphenyl)-N-[(4-methoxyphenyl)methyl]-1,5-naphthyridin-4-amine

To a suspension of Intermediate 6 (130 mg, 0.310 mmol) in 1-methyl-2-pyrrolidinone (2 mL) was added 4-methoxybenzylamine (46 μL). The mixture was heated at 65° C. for 23 h, then re-treated with 4-methoxybenzylamine (46 μL). After a further 5 h the mixture was cooled to ambient temperature, then diluted with EtOAc (10 mL) and DCM (15 mL). Saturated aqueous sodium bicarbonate solution (5 mL) and water (5 mL) were added. The biphase was mixed and separated, then the aqueous layer was extracted with DCM (2×10 mL). The combined extracts were passed through a phase separator and concentrated in vacuo. The resulting crude orange oil was purified by flash column chromatography on silica (gradient elution 0-60% EtOAc/isohexane), then recrystallized from EtOAc/isohexane multiple times, to afford the title compound (48 mg, 35%) as a white crystalline solid. δ_{H} (DMSO-d₆, 300 MHz,) 8.78 (d, J4.6 Hz, 1H), 8.40 (t, J 6.4 Hz, 1H), 7.77 (d, J 4.5 Hz, 1H), 7.45 (d, J 2.0 Hz, 1H), 7.39-7.31 (m, 3H), 7.10 (d, J 8.4 Hz, 1H), 6.94-6.86 (m, 2H), 6.55 (s, 1H), 4.54 (d, J 6.4 Hz, 2H), 3.83 (s, 3H), 3.80 (s, 3H), 3.72 (s, 3H). LCMS (ES+) [M+H]⁺ 436, RT 2.84 minutes (method 2).

### Intermediate 8

### 2-Chloro-8-(3,4-dimethoxyphenyl)-N-[(2-methylpyridin-4-yl)methyl]-1,5-naphthyridin-4-amine

To a suspension of Intermediate 6 (150 mg, 0.36 mmol) in 1-methyl-2-pyrrolidinone (2.5 mL) was added (2-methylpyridin-4-yl)methanamine (100 μL, 0.72 mmol). The mixture was heated at 80° C. for 21 h, then cooled to ambient temperature. Water (10 mL), saturated aqueous sodium bicarbonate solution (5 mL), EtOAc (20 mL) and DCM (5 mL) were added. The biphase was separated and the aqueous layer was extracted with EtOAc (15 mL). The combined organic layers were washed with 50% saturated aqueous sodium chloride solution (2×20 mL), then dried with anhydrous sodium sulfate and concentrated in vacuo. The residue was purified by flash column chromatography on silica (gradient elution with 0-100% EtOAc/isohexane, followed by 0-10% MeOH/EtOAc) to afford the title compound (93 mg, 55%) as a cream solid. δ_{H} (DMSO-d₆, 300 MHz) 8.82 (d, J 4.5 Hz, 1H), 8.53 (t, J 6.3 Hz, 1H), 8.37 (dd, J 5.1, 0.8 Hz, 1H), 7.80 (d, J 4.6 Hz, 1H), 7.47 (d, J 2.1 Hz, 1H), 7.36 (dd, J 8.4, 2.1 Hz, 1H), 7.23 (s, 1H), 7.16 (dd, J 5.2, 1.6 Hz, 1H), 7.11 (d, J 8.5 Hz, 1H), 6.49 (s, 1H), 4.63 (d, J 6.6 Hz, 2H), 3.84 (s, 3H), 3.81 (s, 3H), 2.43 (s, 3H). LCMS (ES+) [M+H]⁺ 421, RT 2.14 minutes (method 2).

### Intermediate 9

### Ethyl 4-{8-(3,4-dimethoxyphenyl)-4-[(4-methoxyphenyl)methylamino]-1,5-naphthyridin-2-yl}piperazine-1-carboxylate

To Intermediate 7 (40 mg, 0.092 mmol) and 1-methyl-2-pyrrolidinone (0.75 mL) were added 1,8-diazabicyclo[5.4.0]undec-7-ene (28 μL, 0.19 mmol) and ethyl piperazine-1-carboxylate (27 μL, 0.18 mmol). The mixture was heated at 180° C. for 18 h, then cooled to ambient temperature. The crude mixture was diluted with DCM (10 mL), EtOAc (10 mL) and water (10 mL). After separation, the aqueous layer was extracted with DCM (2×10 mL). The combined extracts were washed with 50% saturated aqueous sodium chloride solution (2×10 mL), then passed through a phase separator. The mixture was concentrated in vacuo. The crude material was purified using flash column chromatography on silica (gradient elution with 0-50% EtOAc/isohexane) to afford the title compound (48 mg, 84%) as a yellow oil. δ_{H} (CDCl₃, 300 MHz) 8.43 (d, J 4.5 Hz, 1H), 7.55 (d, J 2.0 Hz, 1H), 7.46 (d, J 4.5 Hz, 1H), 7.41-7.32 (m, 3H), 6.99 (d, J 8.4 Hz, 1H), 6.96-6.83 (m, 3H), 6.03 (s, 1H), 4.48 (d, J 5.5 Hz, 2H), 4.18 (q, J 7.1 Hz, 2H), 3.98 (s, 3H), 3.93 (s, 3H), 3.83 (s, 3H), 3.68-3.49 (m, 8H), 1.29 (t, J 7.1 Hz, 3H). LCMS (ES+) [M+H]⁺ 558, RT 2.06 minutes (method 3).

### Example 1

### (3S)-4-(4-Aminopyrido[3,2-d]pyrimidin-2-yl)-N-(4-methoxy-2-methylphenyl)-3-methyl-piperazine-1-carboxamide

To a solution of Intermediate 1 (525 mg) in 1,4-dioxane (2 mL), maintained at 0° C., was added 4N HCl in 1,4-dioxane. The reaction mixture was stirred at r.t. for 4 h, then concentrated in vacuo and triturated with Et₂O. To a solution of the resulting crude solid (125 mg) in DMF (2 mL), maintained at 0° C., were added DIPEA (0.30 mL, 1.53 mmol) and 1-isocyanato-4-methoxy-2-methylbenzene (100 mg, 0.56 mmol). The reaction mixture was stirred at 0° C. for 1 h, then quenched with H₂O (10 mL). The aqueous layer was extracted with EtOAc (50 mL). The organic layer was washed with H₂O (20 mL) and brine (20 mL), then concentrated in vacuo. The crude residue was purified by column chromatography (Normal Phase; 100-200 mesh silica; 5% MeOH in DCM) to afford the title compound (85 mg, 41%) as a white solid. δ_{H} (DMSO-d₆, 400 MHz) 8.42 (br s, 1H), 7.98 (s, 1H), 7.72-7.64 (m, 1H), 7.55-7.60 (m, 3H), 7.04 (d, J 8.6 Hz, 1H), 6.82-6.80 (m, 1H), 6.78 (dd, J 8.6, 2.8 Hz, 1H), 4.88 (m, 1H), 4.55 (d, J 13.4 Hz, 1H), 4.18 (d, J 13.0 Hz, 1H), 4.00 (d, J 13.0 Hz, 1H), 3.72 (s, 3H), 3.20-3.15 (m, 2H), 3.00-2.95 (m, 1H), 2.15 (s, 3H), 1.23 (d, J 6.6 Hz, 3H). LCMS (ES+) [M+H]⁺ 408.3, RT 1.85 minutes (method 2).

### Example 2

### (3S)-4-(4-Aminopyrido[3,2-d]pyrimidin-2-yl)-N-[6-(3,3-difluoroazetidin-1-yl)-2-methyl-pyridin-3-yl]-3-methylpiperazine-1-carboxamide

To a solution of Intermediate 1 (525 mg) in 1,4-dioxane (2 mL), maintained at 0° C., was added 4N HCl in 1,4-dioxane. The reaction mixture was stirred at r.t. for 4 h, then concentrated in vacuo and triturated with Et₂O. To a solution of the resulting crude solid (125 mg) in DMSO (2 mL), maintained at 0° C., was added DIPEA (0.26 mL, 1.53 mmol). The reaction mixture was stirred for 15 minutes, then phenyl N-[6-(3,3-difluoro-azetidin-1-yl)-2-methylpyridin-3-yl]carbamate (WO 2014/096423 A1) (195 mg, 0.612 mmol) was added. The reaction mixture was stirred at 90° C. for 3 h, then quenched with H₂O (10 mL). The aqueous layer was extracted with EtOAc (50 mL). The organic layer was washed with H₂O (20 mL) and brine (20 mL), then concentrated in vacuo. The crude residue was purified by column chromatography (Normal Phase; 100-200 mesh silica; 5% MeOH in DCM) to afford the title compound (120 mg, 37%) as an off-white solid. δ_{H} (DMSO-d₆, 400 MHz) 8.36 (dd, J 4.0, 1.2 Hz, 1H), 8.04 (s, 1H), 7.65 (d, J 7.4 Hz, 1H), 7.60-7.50 (m, 3H), 7.35 (d, J 8.5 Hz, 1H), 6.39 (d, J 8.5 Hz, 1H), 5.02-4.95 (m, 1H), 4.55 (d, J 13.2 Hz, 1H), 4.33 (t, J 12.5 Hz, 4H), 4.13 (d, J 12.6 Hz, 1H), 3.99 (d, J 13.2 Hz, 1H), 3.20-3.10 (m, 2H), 2.98-2.90 (m, 1H), 2.25 (s, 3H), 1.20 (d, J 6.6 Hz, 3H). LCMS (ES+) [M+H]⁺ 470.3, RT 1.81 minutes (method 2).

### Example 3

### (3S)-4-(4-Aminopyrido[3,2-d]pyrimidin-2-yl)-N-[6-(dimethylamino)-2-methylpyridin-3-yl]-3-methylpiperazine-1-carboxamide

To a solution of Intermediate 1 (525 mg) in 1,4-dioxane (2 mL), maintained at 0° C., was added 4N HCl in 1,4-dioxane. The reaction mixture was stirred at r.t. for 4 h, then concentrated in vacuo and triturated with Et₂O. The resulting crude solid (125 mg) and Intermediate 2 (160 mg, 0.612 mmol) were combined using the method described for Example 2 to give the title compound (100 mg, 46%) as an off-white solid. 8_{H} (DMSO-d₆, 400 MHz) 8.35 (dd, J 4.1, 1.3 Hz, 1H), 7.93 (s, 1H), 7.69-7.61 (m, 1H), 7.60-7.50 (m, 3H), 7.21 (d, J 8.7 Hz, 1H), 6.42 (d, J 8.7 Hz, 1H), 4.90-4.85 (m, 1H), 4.56-4.52 (m, 1H), 4.18 (d, J 13.4 Hz, 1H), 3.99 (d, J 13.4 Hz, 1H), 3.18-3.10 (m, 2H), 2.98 (s, 6H), 2.94-2.88 (m, 1H), 2.21 (s, 3H), 1.17 (d, J 6.6 Hz, 3H). LCMS (ES+) [M+H]⁺ 422.3, RT 1.79 minutes (method 2).

### Example 4

### (3S)-4-(4-Aminopyrido[3,2-d]pyrimidin-2-yl)-N-(6-methoxy-2-methylpyridin-3-yl)-3-methylpiperazine-1-carboxamide

To a solution of Intermediate 1 (525 mg) in 1,4-dioxane (2 mL), maintained at 0° C., was added 4N HCl in 1,4-dioxane. The reaction mixture was stirred at r.t. for 4 h, then concentrated in vacuo and triturated with Et₂O. The resulting crude solid (200 mg) and phenyl N-(6-methoxy-2-methylpyridin-3-yl)carbamate (WO 2014/096423 A1) (160 mg, 0.61 mmol) were combined using the method described for Example 2 to give the title compound (65 mg, 28%) as a white solid. δ_{H} (DMSO-d₆, 400 MHz) 8.39-8.31 (m, 1H), 8.10 (s, 1H), 7.65 (d, J 8.4 Hz, 1H), 7.58-7.46 (m, 3H), 7.42 (d, J 8.5 Hz, 1H), 6.60 (d, J 8.5 Hz, 1H), 5.00 (s, 1H), 4.55 (d, J 13.3 Hz, 1H), 4.20-3.90 (m, 2H), 3.32 (s, 3H), 3.20-3.10 (m, 2H), 2.99-2.89 (m, 1H), 2.29 (s, 3H), 1.18 (d, J 6.6 Hz, 3H). LCMS (ES+) [M+H]⁺ 409.4, RT 1.68 minutes (method 2).

### Example 5

### 4-(4-Aminopyrido[3,2-c]pyrimidin-2-yl)-N-(4-methoxy-2-methylphenyl)piperazine-1-carboxamide

To a solution of 2-chloropyrido[3,2-d]pyrimidin-4-amine (J. Chem. Soc., 1956, 1045-54) (300 mg, 1.66 mmol) in n-BuOH (4 mL) was added tert-butyl piperazine-1-carboxylate (370 mg, 1.99 mmol), followed by DIPEA (0.9 mL, 4.99 mmol). The reaction mixture was heated at 130° C. for 16 h, then concentrated in vacuo and diluted with EtOAc (50 mL). The organic layer was washed with H₂O (15 mL) and brine, then dried over anhydrous Na₂SO₄ and concentrated in vacuo. The crude residue was purified by column chromatography (Normal Phase; 100-200 mesh silica; 20% EtOAc in hexanes). The residue was taken up in 1,4-dioxane (2 mL), maintained at 0° C., and 4N HCl in 1,4-dioxane (2 mL) was added. The reaction mixture was stirred at r.t. for 4 h, then concentrated in vacuo and triturated with Et₂O. The resulting crude solid (200 mg) and 1-isocyanato-4-methoxy-2-methylbenzene (147 mg, 0.90 mmol) were combined using the method described for Example 1 to give the title compound (90 mg, 30%) as a white solid. δ_{H} (DMSO-d₆, 400 MHz) 8.37 (d, J 3.9 Hz, 1H), 8.00 (s, 1H), 7.66 (d, J 8.5 Hz, 1H), 7.60-7.45 (m, 3H), 7.04 (d, J 8.6 Hz, 1H), 6.77 (s, 1H), 6.70 (d, J 8.5 Hz, 1H), 3.86-3.80 (m, 4H), 3.72 (s, 3H), 3.54-3.42 (m, 4H), 2.14 (s, 3H). LCMS (ES+) [M+H]⁺ 394, RT 1.73 minutes (method 2).

### Example 6

### Ethyl 4-{8-(3,4-dimethoxyphenyl)-4-[(2-methylpyridin-4-yl)methylamino]-1,5-naphthyridin-2-yl}piperazine-1-carboxylate

To Intermediate 8 (90 mg, 0.19 mmol) were added 1-methyl-2-pyrrolidinone (1.5 mL), 1,8-diazabicyclo[5.4.0]undec-7-ene (57 μL, 0.38 mmol) and ethyl piperazine-1-carboxylate (56 μL, 0.38 mmol). The reaction mixture was heated at 180° C. After 3 h, ethyl piperazine-1-carboxylate (56 μL, 0.38 mmol) was added and the mixture was heated at 160° C. for 1.5 h. The mixture was cooled, then diluted with EtOAc (10 mL), DCM (10 mL), water (10 mL) and saturated aqueous sodium chloride solution (10 mL). The biphase was separated and the aqueous phase was extracted with DCM (10 mL). The combined extracts were passed through a phase separator, then concentrated in vacuo. The residue was purified using reverse phase flash column chromatography on C18 reverse phase silica (pH 10, gradient elution with 0-100% acetonitrile/water), then by preparative HPLC, to afford the title compound (6 mg, 6%) as an off-white solid. δ_{H} (DMSO-d₆, 400 MHz) 8.47 (d, J 4.5 Hz, 1H), 8.37 (d, J 5.1 Hz, 1H), 7.80 (t, J 6.6 Hz, 1H), 7.57-7.54 (m, 2H), 7.38 (dd, J 8.3, 2.0 Hz, 1H), 7.26 (s, 1H), 7.19 (d, J 5.2 Hz, 1H), 7.08 (d, J 8.5 Hz, 1H), 6.12 (s, 1H), 4.60 (d, J 6.5 Hz, 2H), 4.06 (q, J 7.1 Hz, 2H), 3.83 (s, 3H), 3.79 (s, 3H), 3.58-3.52 (m, 4H), 3.45-3.38 (m, 4H), 2.43 (s, 3H), 1.20 (t, J 7.1 Hz, 3H). LCMS (ES+) [M+H]⁺ 543, RT 2.40 minutes (method 2).

### Example 7

### 1-(4-{8-(3,4-Dimethoxyphenyl)-4-[(2-methylpyridin-4-yl)methylamino]-1,5-naphthyridin-2-yl}piperazin-1-yl)ethanone

To a suspension of Intermediate 8 (90 mg, 0.16 mmol) in 1,4-dioxane (2 mL) were added 1-acetylpiperazine (41 mg, 0.32 mmol), cesium carbonate (130 mg, 0.40 mmol) and bis(tri-tert-butylphosphine)palladium(0) (16 mg, 0.032 mmol). The mixture was purged with nitrogen gas for 10 minutes before being heated at 100° C. for 42 h. The mixture was cooled, concentrated in vacuo and taken up in DCM (10 mL). The mixture was washed with water (10 mL) and the aqueous phase was extracted with DCM (2×10 mL). The combined extracts were dry-loaded onto silica and the crude material was purified using flash column chromatography on silica (gradient elution with 0-100% EtOAc in isohexane, followed by 0-20% MeOH/EtOAc). Further purification by preparative HPLC afforded the title compound (17 mg, 21%) as a white solid. δ_{H} (DMSO-d₆, 300 MHz) 8.46 (d, J 4.5 Hz, 1H), 8.36 (d, J 5.1 Hz, 1H), 7.80 (t, J 6.6 Hz, 1H), 7.57-7.53 (m, 2H), 7.36 (dd, J 8.4, 2.0 Hz, 1H), 7.26 (s, 1H), 7.21-7.15 (m, 1H), 7.07 (d, J 8.5 Hz, 1H), 6.11 (s, 1H), 4.60 (d, J 6.5 Hz, 2H), 3.82 (s, 3H), 3.79 (s, 3H), 3.62-3.54 (m, 2H), 3.53-3.43 (m, 6H), 2.43 (s, 3H), 2.01 (s, 3H). LCMS (ES+) [M+H]⁻ 513, RT 2.18 minutes (method 4).

### Example 8

### 1-{4-[4-Amino-8-(3,4-dimethoxyphenyl)-1,5-naphthyridin-2-yl]piperazin-1-yl}ethanone

To a suspension of Intermediate 7 (48 mg, 0.11 mmol) in 1-methyl-2-pyrrolidinone (0.75 mL) were added 1-acetylpiperazine (28 mg, 0.22 mmol) and 1,8-diazabicyclo[5.4.0]undec-7-ene (33 μL, 0.22 mmol). The mixture was heated at 180° C. for 20 h. 1-Acetylpiperazine (0.1 mL, 0.79 mmol) was added and heating was continued for 7 h. The reaction mixture was cooled, then purified using flash column chromatography on silica (gradient elution with 0-100% EtOAc/isohexane, followed by 0-100% MeOH/EtOAc). The isolated material was treated with TFA (5 mL) and stirred at ambient temperature for 70 h. The mixture was concentrated in vacuo. Purification using flash column chromatography on C18 reverse phase silica (pH 10, gradient elution with 0-100% acetonitrile/water), followed by preparative HPLC, afforded the title compound (13 mg, 29%) as a white solid. δ_{H} (DMSO-d₆, 300 MHz) 8.42 (d, J 4.5 Hz, 1H), 7.57 (d, J 2.0 Hz, 1H), 7.51 (d, J 4.5 Hz, 1H), 7.36 (dd, J 8.4, 2.0 Hz, 1H), 7.07 (d, J 8.5 Hz, 1H), 6.52 (s, 2H), 6.39 (s, 1H), 3.83 (s, 3H), 3.80 (s, 3H), 3.65-3.49 (m, 8H), 2.04 (s, 3H). LCMS (ES+) [M+H]⁻ 408, RT 1.86 minutes (method 4).

### Example 9

### Ethyl 4-[4-amino-8-(3,4-dimethoxyphenyl)-1,5-naphthyridin-2-yl]piperazine-1-carboxylate

To Intermediate 9 (45 mg, 0.08 mmol) was added TFA (1 mL). The mixture was stirred at ambient temperature for 24 h before being re-treated with TFA (1 mL), then stirred for a further 24 h. The mixture was concentrated in vacuo. The resulting orange gum was purified using preparative HPLC to afford the title compound (17 mg, 50%) as a white solid. δ_{H} (DMSO-d₆, 300 MHz) 8.42 (d, J 4.5 Hz, 1H), 7.57 (d, J 2.0 Hz, 1H), 7.52 (d, J 4.5 Hz, 1H), 7.38 (dd, J 8.4, 2.0 Hz, 1H), 7.07 (d, J 8.5 Hz, 1H), 6.52 (br s, 2H), 6.39 (s, 1H), 4.07 (q, J 7.1 Hz, 2H), 3.83 (s, 3H), 3.81 (s, 3H), 3.61-3.52 (m, 4H), 3.51-3.42 (m, 4H), 1.20 (t, J 7.1 Hz, 3H). LCMS (ES+) [M+H]⁺ 438, RT 2.15 minutes (method 2).

1. A compound of formula (I) or an N-oxide thereof, or a pharmaceutically acceptable salt or solvate thereof: 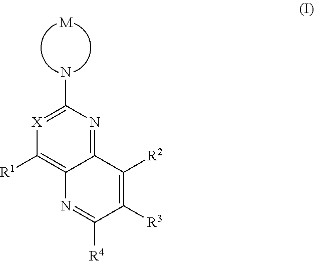
wherein
X represents N or CH;
M represents the residue of an optionally substituted saturated four-, five-, six- or seven-membered monocyclic ring containing one nitrogen atom and 0, 1, 2 or 3 additional heteroatoms independently selected from N, O and S, but containing no more than one O or S atom; or
M represents the residue of an optionally substituted saturated or unsaturated 5- to 10-membered fused bicyclic ring system containing one nitrogen atom and 0, 1, 2 or 3 additional heteroatoms independently selected from N, O and S, but containing no more than one O or S atom; or
M represents the residue of an optionally substituted saturated 5- to 9-membered bridged bicyclic ring system containing one nitrogen atom and 0, 1, 2 or 3 additional heteroatoms independently selected from N, O and S, but containing no more than one O or S atom; or
M represents the residue of an optionally substituted saturated 5- to 9-membered spirocyclic ring system containing one nitrogen atom and 0, 1, 2 or 3 additional heteroatoms independently selected from N, O and S, but containing no more than one O or S atom;
R¹, R² and R³ independently represent hydrogen, halogen, cyano, nitro, hydroxy, trifluoromethyl, trifluoromethoxy, —SR^{a}, —SR^{a}, —SOR^{a}, —SO₂R^{a}, —NR^{b}R^{c}, —CH₂NR^{b}R^{c}, —NR^{c}COR^{d}, —CH₂NR^{c}COR^{d}, —NR^{c}CO₂R^{d}, —NHCONR^{b}R^{c}, —NR^{c}SO₂R^{e}, —N(SO₂R^{e})₂, —NHSO₂NR^{b}R^{c}, —COR^{d}, —CO₂R^{d}, —CONR^{b}R^{c}, —CON(OR^{a})R^{b} or —SO₂NR^{b}R^{c}; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, C₃₋₇ heterocycloalkenyl, heteroaryl or heteroaryl(C₁₋₆)alkyl, any of which groups may be optionally substituted by one or more substituents;
R⁴ represents hydrogen, halogen, cyano, trifluoromethyl or C₁₋₆ alkyl;
R^{a} represents hydrogen; or R^{a} represents C₁₋₆ alkyl, aryl, aryl(C₁₋₆)alkyl, heteroaryl or heteroaryl(C₁₋₆)alkyl, any of which groups may be optionally substituted by one or more substituents;
R^{b} and R^{c} independently represent hydrogen or trifluoromethyl; or C₁₋₆ alkyl, C₃₋₇ cycloalkyl, C₃₋₇ cycloalkyl(C₁₋₆)alkyl, aryl, aryl(C₁₋₆)alkyl, C₃₋₇ heterocycloalkyl, C₃₋₇ heterocycloalkyl(C₁₋₆)alkyl, heteroaryl or heteroaryl(C₁₋₆)alkyl, any of which groups may be optionally substituted by one or more substituents; or
R^{b} and R^{c}, when taken together with the nitrogen atom to which they are both attached, represent azetidin-1-yl, pyrrolidin-1-yl, oxazolidin-3-yl, isoxazolidin-2-yl, thiazolidin-3-yl, isothiazolidin-2-yl, piperidin-1-yl, morpholin-4-yl, thiomorpholin-4-yl, piperazin-1-yl, homopiperidin-1-yl, homomorpholin-4-yl or homopiperazin-1-yl, any of which groups may be optionally substituted by one or more substituents;
R^{d} represents hydrogen; or C₁₋₆ alkyl, C₃₋₇ cycloalkyl, aryl, C₃₋₇ heterocycloalkyl or heteroaryl, any of which groups may be optionally substituted by one or more substituents; and
R^{e} represents C₁₋₆ alkyl, aryl or heteroaryl, any of which groups may be optionally substituted by one or more substituents.

2. The compound as claimed in claim 1 wherein R¹ represents —NR^{b}R^{c}, in which R^{b} and R^{c} are as defined in claim 1.

3. The compound as claimed in claim 1 represented by formula (IIA), or a pharmaceutically acceptable salt or solvate thereof:

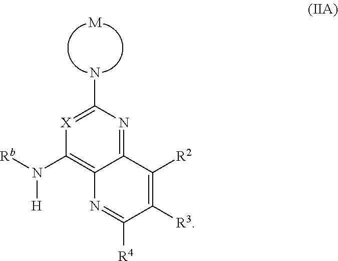

4. The compound as claimed in claim 1 wherein M represents the residue of a piperazin-1-yl ring, optionally substituted by one or two substituents independently selected from C₁₋₆ alkyl, C₂₋₆ alkylcarbonyl, C₂₋₆ alkoxy-carbonyl, (C₁₋₆ alkoxy)(C₁₋₆ alkyl)phenylaminocarbonyl, (C₁₋₆ alkoxy)(C₁₋₆ alkyl)-pyridinylaminocarbonyl, [di(C₁₋₆)alkylamino](C₁₋₆ alkyl)pyridinylaminocarbonyl and (dihaloazetidinyl)(C₁₋₆ alkyl)pyridinylaminocarbonyl.

5. The compound of formula (I) as defined in claim 1 as herein specifically disclosed in any one of the Examples.

6. (canceled)

7. (canceled)

8. A pharmaceutical composition comprising a compound of formula (I) as defined in claim 1 or an N-oxide thereof, or a pharmaceutically acceptable salt or solvate thereof, in association with a pharmaceutically acceptable carrier.

9. (canceled)

10. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 1 or an N-oxide thereof, or a pharmaceutically acceptable salt or solvate thereof.

11. The compound as claimed in claim 2 represented by formula (IIA), or a pharmaceutically acceptable salt or solvate thereof:

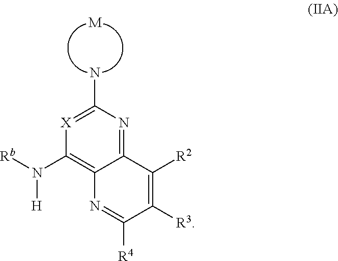

12. The compound as claimed in claim 2 wherein M represents the residue of a piperazin-1-yl ring, optionally substituted by one or two substituents independently selected from C₁₋₆ alkyl, C₂₋₆ alkylcarbonyl, C₂₋₆ alkoxy-carbonyl, (C₁₋₆ alkoxy)(C₁₋₆ alkyl)phenylaminocarbonyl, (C₁₋₆ alkoxy)(C₁₋₆ alkyl)-pyridinylaminocarbonyl, [di(C₁₋₆)alkylamino](C₁₋₆ alkyl)pyridinylaminocarbonyl and (dihaloazetidinyl)(C₁₋₆ alkyl)pyridinylaminocarbonyl.

13. The compound as claimed in claim 3 wherein M represents the residue of a piperazin-1-yl ring, optionally substituted by one or two substituents independently selected from C₁₋₆ alkyl, C₂₋₆ alkylcarbonyl, C₂₋₆ alkoxy-carbonyl, (C₁₋₆ alkoxy)(C₁₋₆ alkyl)phenylaminocarbonyl, (C₁₋₆ alkoxy)(C₁₋₆ alkyl)-pyridinylaminocarbonyl, [di(C₁₋₆)alkylamino](C₁₋₆ alkyl)pyridinylaminocarbonyl and (dihaloazetidinyl)(C₁₋₆ alkyl)pyridinylaminocarbonyl.

14. A pharmaceutical composition comprising a compound of formula (I) as defined in claim 3 or an N-oxide thereof, or a pharmaceutically acceptable salt or solvate thereof, in association with a pharmaceutically acceptable carrier.

15. A pharmaceutical composition comprising a compound of formula (I) as defined in claim 3 or an N-oxide thereof, or a pharmaceutically acceptable salt or solvate thereof, in association with a pharmaceutically acceptable carrier.

16. A pharmaceutical composition comprising a compound of formula (I) as defined in claim 4 or an N-oxide thereof, or a pharmaceutically acceptable salt or solvate thereof, in association with a pharmaceutically acceptable carrier.

17. A pharmaceutical composition comprising a compound of formula (I) as defined in claim 11 or an N-oxide thereof, or a pharmaceutically acceptable salt or solvate thereof, in association with a pharmaceutically acceptable carrier.

18. A pharmaceutical composition comprising a compound of formula (I) as defined in claim 12 or an N-oxide thereof, or a pharmaceutically acceptable salt or solvate thereof, in association with a pharmaceutically acceptable carrier.

19. A pharmaceutical composition comprising a compound of formula (I) as defined in claim 13 or an N-oxide thereof, or a pharmaceutically acceptable salt or solvate thereof, in association with a pharmaceutically acceptable carrier.

20. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 3 or an N-oxide thereof, or a pharmaceutically acceptable salt or solvate thereof.

21. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 5 or an N-oxide thereof, or a pharmaceutically acceptable salt or solvate thereof.

22. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 11 or an N-oxide thereof, or a pharmaceutically acceptable salt or solvate thereof.

23. A method for the treatment and/or prevention of an inflammatory, autoimmune or oncological disorder, a viral disease or malaria, or organ or cell transplant rejection, which comprises administering to a patient in need of such treatment an effective amount of a compound of formula (I) as defined in claim 13 or an N-oxide thereof, or a pharmaceutically acceptable salt or solvate thereof.
